SEQUENCE LISTING (54 sequences)

SEQ ID NO: 1 - protein, 5 aa, Artificial Sequence
  Generic DAT-C5 variants
  MISC_FEATURE (1)..(1): H, N, F, or T; or is absent;
  MISC_FEATURE (2)..(2): W, S, E, or Y; or is absent;
  MISC_FEATURE (3)..(3): L, V, or I;
  MISC_FEATURE (4)..(4): K, I, or R;
  MISC_FEATURE (5)..(5): V;
XXXXX

SEQ ID NO: 2 - protein, 5 aa, Artificial Sequence
  Generic DAT-C5 variants
  MISC_FEATURE (1)..(1): N, F, or T; or is absent;
  MISC_FEATURE (2)..(2): S, E, or Y; or is absent;
  MISC_FEATURE (3)..(3): V, L, or I;
  MISC_FEATURE (4)..(4): I, or R;
  MISC_FEATURE (5)..(5): V;
XXXXX

SEQ ID NO: 3 - protein, 5 aa, Artificial Sequence
  Generic DAT-C5 variants
  MISC_FEATURE (1)..(1): N, or T; or is absent
  MISC_FEATURE (2)..(2): S, E, or Y; or is absent;
  MISC_FEATURE (3)..(3): V, L, or I;
  MISC_FEATURE (4)..(4): I, or R;
  MISC_FEATURE (5)..(5): V;
XXXXX

SEQ ID NO: 4 - protein, 5 aa, Artificial Sequence
  Generic DAT-C5 variants
  MISC_FEATURE (1)..(1): N, or F; or is absent;
  MISC_FEATURE (2)..(2): S, E, or Y; or is absent;
  MISC_FEATURE (3)..(3): V, L, or I;
  MISC_FEATURE (4)..(4): I, or R;
  MISC_FEATURE (5)..(5): V;
XXXXX

SEQ ID NO: 5 - protein, 5 aa, Artificial Sequence
  DAT-C5 variant
NSIIV

SEQ ID NO: 6 - protein, 5 aa, Artificial Sequence
  DAT-C5 variant
NSIRV

SEQ ID NO: 7 - protein, 5 aa, Artificial Sequence
  DAT-C5 variant
NSVIV

SEQ ID NO: 8 - protein, 5 aa, Artificial Sequence
  DAT-C5 variant
NSVRV

SEQ ID NO: 9 - protein, 5 aa, Artificial Sequence
  DAT-C5 variant
NEIIV

SEQ ID NO: 10 - protein, 5 aa, Artificial Sequence
  DAT-C5 variant
NEIRV

SEQ ID NO: 11 - protein, 5 aa, Artificial Sequence
  DAT-C5 variant
NEVIV

SEQ ID NO: 12 - protein, 5 aa, Artificial Sequence
  DAT-C5 variant
NEVRV

SEQ ID NO: 13 - protein, 5 aa, Artificial Sequence
  DAT-C5 variant
NYIIV

SEQ ID NO: 14 - protein, 5 aa, Artificial Sequence
  DAT-C5 variant
NYIRV

SEQ ID NO: 15 - protein, 5 aa, Artificial Sequence
  DAT-C5 variant
NYVIV

SEQ ID NO: 16 - protein, 5 aa, Artificial Sequence
  DAT-C5 variant
NYVRV

SEQ ID NO: 17 - protein, 5 aa, Artificial Sequence
  DAT-C5 variant
TSIIV

SEQ ID NO: 18 - protein, 5 aa, Artificial Sequence
  DAT-C5 variant
TSIRV

SEQ ID NO: 19 - protein, 5 aa, Artificial Sequence
  DAT-C5 variant
TSVIV

SEQ ID NO: 20 - protein, 5 aa, Artificial Sequence
  DAT-C5 variant
TSVRV

SEQ ID NO: 21 - protein, 5 aa, Artificial Sequence
  DAT-C5 variant
TEIIV

SEQ ID NO: 22 - protein, 5 aa, Artificial Sequence
  DAT-C5 variant
TEIRV

SEQ ID NO: 23 - protein, 5 aa, Artificial Sequence
  DAT-C5 variant
TEVIV

SEQ ID NO: 24 - protein, 5 aa, Artificial Sequence
  DAT-C5 variant
TEVRV

SEQ ID NO: 25 - protein, 16 aa, Artificial Sequence
  DAT-C5 variant
DATCVARIANTTYIIV

SEQ ID NO: 26 - protein, 5 aa, Artificial Sequence
  DAT-C5 variant
TYIRV

SEQ ID NO: 27 - protein, 5 aa, Artificial Sequence
  DAT-C5 variant
TYVIV

SEQ ID NO: 28 - protein, 5 aa, Artificial Sequence
  DAT-C5 variant
TYVRV

SEQ ID NO: 29 - protein, 5 aa, Artificial Sequence
  DAT-C5 variant
FSIIV

SEQ ID NO: 30 - protein, 5 aa, Artificial Sequence
  DAT-C5 variant
FSIRV

SEQ ID NO: 31 - protein, 5 aa, Artificial Sequence
  DAT-C5 variant
FSVIV

SEQ ID NO: 32 - protein, 5 aa, Artificial Sequence
  DAT-C5 variant
FSVRV

SEQ ID NO: 33 - protein, 5 aa, Artificial Sequence
  DAT-C5 variant
FEIIV

SEQ ID NO: 34 - protein, 5 aa, Artificial Sequence
  DAT-C5 variant
FEIRV

SEQ ID NO: 35 - protein, 5 aa, Artificial Sequence
  DAT-C5 variant
FEVIV

SEQ ID NO: 36 - protein, 5 aa, Artificial Sequence
  DAT-C5 variant
FEVRV

SEQ ID NO: 37 - protein, 5 aa, Artificial Sequence
  DAT-C5 variant
FYIIV

SEQ ID NO: 38 - protein, 5 aa, Artificial Sequence
  DAT-C5 variant
FYIRV

SEQ ID NO: 39 - protein, 5 aa, Artificial Sequence
  DAT-C5 variant
FYVIV

SEQ ID NO: 40 - protein, 5 aa, Artificial Sequence
  DAT-C5 variant
FYVRV

SEQ ID NO: 41 - protein, 4 aa, Artificial Sequence
  DAT-C5 variant
SIIV

SEQ ID NO: 42 - protein, 4 aa, Artificial Sequence
  DAT-C5 variant
SIRV

SEQ ID NO: 43 - protein, 4 aa, Artificial Sequence
  DAT-C5 variant
SVIV

SEQ ID NO: 44 - protein, 4 aa, Artificial Sequence
  DAT-C5 variant
SVRV

SEQ ID NO: 45 - protein, 4 aa, Artificial Sequence
  DAT-C5 variant
EIIV

SEQ ID NO: 46 - protein, 4 aa, Artificial Sequence
  DAT-C5 variant
EIRV

SEQ ID NO: 47 - protein, 4 aa, Artificial Sequence
  DAT-C5 variant
EVIV

SEQ ID NO: 48 - protein, 4 aa, Artificial Sequence
  DAT-C5 variant
EVRV

SEQ ID NO: 49 - protein, 4 aa, Artificial Sequence
  DAT-C5 variant
YIIV

SEQ ID NO: 50 - protein, 4 aa, Artificial Sequence
  DAT-C5 variant
YIRV

SEQ ID NO: 51 - protein, 4 aa, Artificial Sequence
  DAT-C5 variant
YVIV

SEQ ID NO: 52 - protein, 4 aa, Artificial Sequence
  DAT-C5 variant
YVRV

SEQ ID NO: 53 - protein, 5 aa, Artificial Sequence
  DAT-C5 variant
NSLRV

SEQ ID NO: 54 - protein, 5 aa, Artificial Sequence
  DAT-C5 variant
HWLKV

(19) United States
(12) Patent Application Publication
(10) Pub. No.: US 2023/0346948 A1
(43) Pub. Date: Nov. 2, 2023

(54) Lipid conjugated peptide inhibitors of PICK1

(71) Applicant: University of Copenhagen, Copenhagen K (DK)

(72) Inventors: Kenneth L. Madsen, Virum (DK); Ulrik Gether, Charlottenlund (DK); Nikolaj Riis Christensen, Allerød (DK)

(21) Appl. No.: 17/905,660

(22) PCT Filed: Mar. 5, 2021
(86) PCT No.: PCT/EP2021/055678

§ 371 Date: Sep. 6, 2022

(30) Foreign Application Priority Data
Mar. 6, 2020 (EP) 20161493.0

(51) Int. Cl. A61K 47/54; A61K 47/60; A61K 38/08; A61P 25/00; A61K 47/69

(52) U.S. Cl. CPC A61K 47/542; A61K 47/60; A61K 38/08; A61P 25/00; A61K 47/6907

(57) ABSTRACT

The present disclosure relates to a lipid conjugated bivalent peptide ligand which bind to Protein Interacting with C Kinase-1 (PICK1) and thereby inhibit PICK1. The PICK1 inhibitors of the present disclosure comprise a peptide portion comprising two peptide ligands of PICK1, and a non-peptide portion comprising a linker, linking the two peptide ligands, and a lipid. The disclosure furthermore relates to therapeutic and diagnostic use of said PICK1 inhibitor for treatment of diseases or disorders associated with maladaptive plasticity.

### TECHNICAL FIELD

The present invention relates to a lipid conjugated bivalent peptide ligand which bind to Protein Interacting with C Kinase-1 (PICK1) and thereby inhibit PICK1. The invention furthermore relates to therapeutic and diagnostic use of said PICK1 inhibitor.

### BACKGROUND

Synaptic plasticity serves as the molecular substrate for learning and memory. In the glutamatergic synapse release of Glu activates in particular the N-methyl-Daspartate receptors (NMDARs) and the α-amino-3-hydroxy-5-methyl-4-isoxazole-propionic acid receptors (AMPARs), both ligand-gated ion-channels. Activation of these receptors allows for an influx of Na+ in AMPARs and Ca2+ in the case of NMDARs. In diseased states, such as ischemia after stroke and head injury, neuropathic pain and addiction, abnormal synaptic stimulation causes maladaptive plasticity leading to hyper-sensitization of glutamatergic synapses through expression of calcium permeable (CP) AMPA-type glutamate receptors (CP-AMPARs).

Although numerous disease states involve an over-activation or hyper-sensitization of the glutamate system, the only currently used drugs which target the glutamate system are the NMDA receptor antagonists such as ketamine, which are used as anaesthetics. Development of new glutamate system targeting drugs have proven difficult due to general problems with severe side effects. Diseases such as neuropathic pain, excitotoxicity following ischemia and drug addiction are currently without any effective therapy. Accordingly, there is a need for a treatment of these diseases.

Protein Interacting with C Kinase-1 (PICK1) is a PDZ domain containing scaffolding protein that plays a central role in synaptic plasticity. PICK1 is essential for AM PAR function, mainly through control of AMPAR trafficking. The PDZ domain of PICK1 interacts directly with the C-terminus of the GluA2 subunit of the AMPA receptors (AMPAR) as well as protein kinase A and C, thereby regulating AM PAR phosphorylation and surface expression and in turn synaptic plasticity by tuning the efficacy of individual synapses. PICK1 is an intracellular scaffold protein primarily involved in regulation of protein trafficking and cell migration by mediating and facilitating protein-protein interactions (PPIs) via its PDZ domain. Central to PICK1's cellular role is its ability to bind and interact with numerous intracellular molecules including various protein partners, as well as membrane phospholipids. PICK1 is a functional dimer, with two PDZ domains flanking the central membrane binding BAR domain, which mediates the dimerization.

Protein-protein interactions (PPIs) are vital for most biochemical and cellular processes and are often mediated by scaffold and signal transduction complexes. One of the most abundant classes of human facilitators of PPIs is the family of postsynaptic density protein-95 (PSD-95)/Discs-large/ZO-1 (PDZ) domains. PDZ domain proteins, such as PICK1, in the postsynaptic density dynamically regulate the surface expression and activity of the glutamate receptors and therefore represent attractive drug targets for treatment of diseases or disorders associated with maladaptive plasticity. It has, however, proven challenging to develop sufficiently potent inhibitors for these targets.

Targeting of the PDZ domains of PSD-95 has been successfully attempted by using bivalent peptide ligands. PSD-95 comprises more PDZ domains, including PDZ1 and PDZ2 which share ligand preference, leading to the idea of targeting both PDZ domains with bivalent ligands. The first bivalent inhibitor was suggested by Long, et al. (2003), resulting in only modest affinity towards PSD-95 PDZ12. More successful bivalent peptide ligands have later been developed (Bach et al. 2009).

The dimeric peptide ligand targeting PSD-95, was functionalized with a fatty acid. The modification was found to provide improved plasma half-life and subcutaneous stability of the peptide with no influence on the affinity towards the PDZ domain (WO 2015/078477).

As described above, there is a high need for providing potent inhibitors of PICK1-PDZ domain for treatment of disease or disorder associated with maladaptive plasticity.

### SUMMARY

The present invention provides a high affinity peptide inhibitor towards Protein Interacting with C Kinase-1 (PICK1). The inventors have surprisingly found that by attaching of a lipid to a bivalent peptide ligand of PICK1, a significant increase in potency may be obtained. Such increase in potency is highly important to provide potent inhibitors of PICK1-PDZ domain which are required for development of treatment of disease or disorder associated with maladaptive plasticity. Without being bound by theory, the increased potency of the lipid conjugated bivalent peptide ligand of the present disclosure is thought to be a result of micellar formation which in turn results in formation of higher oligomeric constructs of PICK1 upon binding and thereby inhibition of PICK1. Such increase in potency could not be foreseen from the current use of lipid conjugation to provide improved pharmacokinetics.

By targeting the scaffolding protein PICK1, which is responsible for AMPA receptor trafficking, the risk of possible side effects of the compound is reduced, compared to targeting the receptor directly. The compounds of the present disclosure will provide treatment for patients with conditions such as neuropathic pain, excitotoxicity following ischemia or drug addiction.

In a first aspect, the present disclosure provides a PICK1 inhibitor comprising a peptide portion and a non-peptide portion, wherein the peptide portion consists of
a) a first peptide comprising an amino acid sequence of the general formula: X₁X₂X₃X₄X₅; and
b) a second peptide comprising an amino acid sequence of the general formula: X₁X₂X₃X₄X₅;

wherein
X₁ is H, N, F, or T, or is absent;
X₂ is W, S, E, or Y; or is absent;
X₃ is L, V, or I;
X₄ is K, I, or R; and
X₅ is V;

and wherein the non-peptide portion comprises:
c) a linker linking the first peptide to the second peptide, and
d) a lipophilic aliphatic group.

In a second aspect, the present disclosure provides a micelle comprising a PICK1 inhibitor comprising
a) a first peptide comprising an amino acid sequence of the general formula: X₁X₂X₃X₄X₅; and
b) a second peptide comprising an amino acid sequence of the general formula: X₁X₂X₃X₄X₅;
wherein:
   X₁ is H, N, F, or T, or is absent;
   X₂ is W, S, E, or Y; or is absent;
   X₃ is L, V, or I;
   X₄ is K, I, or R; and
   X₅ is V;
c) a linker linking the first peptide to the second peptide, and
d) a lipophilic aliphatic group.

In a further aspect, the present disclosure provides a pharmaceutical composition comprising the PICK1 inhibitor or the micelle as disclosed herein.

In a further aspect, the PICK1 inhibitor, the micelle or the pharmaceutical composition as disclosed herein is provided for use as a medicament.

In a further aspect, the present disclosure provides a method of providing prophylaxis and/or treatment of a disease or disorder associated with maladaptive plasticity in a subject, the method comprising administering the PICK1 inhibitor, the micelle or the pharmaceutical composition as disclosed herein.

In a further aspect, a method of diagnosing breast cancer in a subject in need thereof is provided, the method comprising the steps of:
a. obtain a tissue sample from said subject;
b. staining the sample with the PICK1 inhibitor comprising a detectable moiety as disclosed herein;
c. determining the level of PICK1 in the sample; and
d. comparing the level of PICK1 in the sample to a healthy standard,

wherein an increased level of PICK1 in the sample is indicative of said individual having breast cancer.

In a further aspect, a method for predicting the prognosis for a subject suffering from breast cancer is provided, the method comprising the steps of:
a. obtain a tissue sample from said subject;
b. staining the sample with the PICK1 inhibitor comprising a detectable moiety as disclosed herein;
c. determining the level of PICK1 in the sample; and
d. comparing the level of PICK1 in the sample to a healthy standard,

wherein an increased level of PICK1 in the sample is indicative of poor prognosis.

### DESCRIPTION OF DRAWINGS

FIG. 1: Concentration dependent self-assembly of myr-NPEG₄-(HWLKV)₂ shown by Size exclusion chromatography (SEC). NPEG₄-(HWLKV)₂ used for control.

FIG. 2: a) Small angle X-ray scattering analysis using a concentration series of myr-NPEG₄-(HWLKV)₂ confirms self-assembly of myr-NPEG₄-(HWLKV)₂ into micellar structures. b) Pair distance distribution function (PDDF), for different concentrations of myr-NPEG₄-(HWLKV)₂. c) PDDF derived sample parameters. M_{w}/M_{wteo} suggest a higher order assembly of myr-NPEG₄-(HWLKV)₂, in the range of 5-8 individual molecules.

FIG. 3: Binding of oligomeric myr-NPEG₄-(HWLKV)₂ to PICK1. Fluorescence polarization competition binding curves of myr-NPEG₄-(HWLKV)₂ (Ki,app=3.0 nM, SEM interval [2.3-3.8] nM, n=6), HWLKV (Ki,app=6998 nM, SEM interval [4972-9849] nM, n=3) and NPEG₄-(HWLKV)₂ (Ki,app=179 nM, SEM interval [169-189], n=6), using 5FAM-NPEG₄-(HWLKV)₂ (5 nM vs. myr-NPEG₄-(HWLKV)₂) or 5FAM-HWLKV (20 nM vs, HWLKV) as tracer (error bars are shown as SEM of n=3).

FIG. 4: SEC elution profile of PICK1 in absence (grey) or presence (black) of myr-NPEG₄-(HWLKV)₂, in a PICK1:myr-NPEG₄-(HWLKV)₂ molecular ratio of 4:1 respectively. The elution profile clearly indicates formation of higher order oligomers.

FIG. 5: Effect of single amino acid substitutions in DAT C5 (HWLKV) on binding affinity. A library of 95 HWLKV peptides with single amino acids substitutions in position X₁-X₅ of the sequence HWLKV was tested in fluorescence polarization binding in competition with fluorescently labelled HWLKV. Data are given as fold change compared to the reference peptide HWLKV (set to 1) with darker shades indicating increase in affinity (up to 3-fold) and lighter shades indicating reduces affinity. White indicate disruption of binding and crosses indicate insoluble peptides. Peptides shown with % were not soluble in buffer and were dissolved in 10% DMSO.

FIG. 6: Fold affinity change measured using FP competition of a combinatorial peptide library combining single amino acid substitutions from previous single substitution screen. Screen suggests NSVRV/TSIRV as optimal 5-mer sequences, EIRV/YIIV as optimal 4-mer sequences, IIV/IRV as optimal 3-mer sequences. These sequences could not have been predicted from initial 5-mer sequence, HWLKV. x indicates insoluble or non-binding peptides.

FIG. 7: Chemical structure of tested bivalent PICK1 inhibitors without the lipid-residue, but with different PEGx linkers, either linked to the N-terminal amine of HWLKV (PEG₀-(C5)₂, PEG₁-(C5)₂, PEG₂-(C5)₂, PEG₃-(C5)₂, PEG₄-(C5)₂) or linked to the lysine (K) side chain amine of sequence HWLKV (ac-(HWLK_{PEG4}V)₂.

FIG. 8: Fold affinity gain over monomeric C5 (HWLKV) peptide for various PEG linker compounds towards purified PICK1

FIGS. 9a-c: a) Efficacy of myr-NPEG₄-(HWLKV)₂ on acute inflammatory pain. In vivo experiments revealing the ability of myr-NPEG₄-(HWLKV)₂ to relieve evoked pain in the Complete Freund's Adjuvant model of inflammatory pain through multiple administration routes. a) s.c. administration, b) s.c. administration, dose response of 2, 10, and 50 μmol/kg, c) i.t. administration. Dotted curves represent data from the contralateral left hind paw used as internal control of the animal. All data is expressed as mean±SEM. Abbreviations; adm.=administration, BL=baseline, CFA=Complete Freund's Adjuvant, i.pl.=intraplantar, i.t.=intrathecal, s.c.=subcutaneous.

FIG. 10:

Efficacy of myr-NPEG₄-(HWLKV)₂ on neuropathic pain. In vivo experiments revealing the ability of myr-NPEG₄-(HWLKV)₂ to relieve evoked pain in the neuropathic SNI model of pain. Dotted curves represent data from the contralateral left hind paw used as internal control of the animal. All data is expressed as mean±SEM. Abbreviations; PWT=paw withdrawal threshold, SNI=spared nerve injury.

FIG. 11: Mice were subjected to SNI surgery and 9 days post-surgery, a decrease of threshold response to von Frey filaments of ipsilateral hind-paw was confirmed by von Frey filaments, corresponding to neuropathic pain condition. On day 9, mice were treated with 10 μmol/kg NPEG₄-(HWLKV)₂ (gray) without lipidation, which does not elicit a significant increase in pain withdrawal threshold. For comparison, the response to 10 μmol/kg myr-NPEG₄-(HWLKV)₂ from FIG. 10 is shown (dashed black). All data is expressed as mean±SEM. Abbreviations; PWT=paw withdrawal threshold, SNI=spared nerve injury.

FIG. 12: Effect of myr-NPEG₄-(HWLKV)₂ after 1 year of chronic pain. Mice were subjected to SNI surgery and 2 days post-surgery, a decrease of threshold response to von Frey filaments of ipsilateral hind-paw was confirmed by von Frey filaments with once monthly observation for 1 year (data not shown). After 52 weeks, mice were treated with 10 μmol/kg myr-NPEG₄-(HWLKV)₂, which produced a highly significant relief of the PWT at 5 h. All data is expressed as mean±SEM. Abbreviations; PWT=paw withdrawal threshold, SNI=spared nerve injury.

FIG. 13: Efficacy of myr-NPEG₄-(HWLKV)₂ diabetic neuropathy. Mice were given a single IP injection 200 μg/mL Streptozocin solution to induce diabetes (STZ Model) and glycemia is tested before, and 7 days after injection. 13 days post-surgery, a decrease of threshold response to von Frey filaments of ipsilateral hind-paw was confirmed by von Frey filaments, corresponding to diabetic neuropathy. S.c. injection of myr-NPEG₄-(HWLKV)₂ demonstrated a dose dependent increase in PWT with similar efficacy as pregabalin. All data is expressed as mean±SEM. Abbreviations; PWT=paw withdrawal threshold, STZ=streptozocin.

FIG. 14: Efficacy of variants of the PDZ binding motif. Mice were injected i.pl. into the right hind paw with 50 μL of CFA. On day 2 after CFA injection, mice were injected s.c. with 0.4 μmol/kg and on day 5, with 2 μmol/kg of myr-NPEG₄ peptides with the C5 sequence substitutes as indicated. Treatment with 0.4 μmol/kg myr-NPEG₄-(NSVRV)₂ significantly increased PWT, while 2 μmol/kg of myr-NPEG₄-(HWLKV)₂ and myr-NPEG₄-(SVRV)₂ significantly increased PWT. At 2 μmol/kg, myr-NPEG₄-(NSVRV)₂ was not well dissolved. All data is expressed as mean±SEM. Abbreviations; PWT=paw withdrawal threshold, CFA=complete Freuds Adjuvans.

FIG. 15: Efficacy of myr-NPEG₄-(HWLKV)₂ in relief of spontaneous pain. Mice were injected i.pl. into the right hind paw with 50 μL of CFA prior to a single injection of saline in the striped chamber and myr-NPEG₄-(HWLKV)₂ (30 μmol/kg) in the gray chamber of the apparatus illustrated on the left. On a separate day, the preference for the chambers was determined by the time spend in each chamber. CFA injected mice spend significantly increased amount of time in the chamber where they were injected with myr-NPEG₄-(HWLKV)₂ demonstrating relief of spontaneous pain. Naïve mice (not treated with CFA) did not show place preference to a single administration of myr-NPEG₄-(HWLKV)₂.

FIG. 16: Dose-dependent plasma exposure of myr-NPEG₄-(HWLKV)₂. myr-NPEG₄-(HWLKV)₂ was administered S.c. to mice in three different doses as indicated and plasma exposure at different times determined by LC-MS. Plasma concentrations peak in a dose-dependent manner at 1 h post injection and decrease with linear kinetics, but show no increase in life-time compared to the non-lipidated peptide Tat-NPEG₄-(HWLKV)₂.

FIG. 17: Solubility of myr-NPEG₄-(HWLKV)₂. Photograph of myr-NPEG₄-(HWLKV)₂ solubilized in PBS at 130 mM (250 mg/ml).

FIG. 18: Results from efficacy testing of different compounds of the invention in an animal model of inflammatory pain. Mice were injected i.pl. into the right hind paw with 50 μL of CFA. On day 2-5 after CFA injection, mice were injected s.c. with 2 μmol/kg of X-NPEG₄-(HWLKV)₂ with the lipid substitutes (X) as indicated. All data is expressed as mean±SEM. Abbreviations; PWT=paw withdrawal threshold, CFA=complete Freuds Adjuvans.
A: Unsaturated fatty acids and fatty diacid.
B: Fatty acid lengths.
C: Amino acid adducts and cholesterol.

FIG. 19: Efficacy of mPD5 on thermal hypersensitivity. Mice were injected i.pl. into the right hind paw with 50 μL of CFA. On day 3 after CFA injection, a reduction in PWL to stimulation with a laser beam in Hargreaves test confirmed thermal hypersensitivity. Mice were injected s.c. with 10 μmol/kg (10 μl PBS/g) of myr-NPEG₄-(HWLKV)₂ (mPD5), giving rise to a significant increase in PWL in the CFA injected paw (ipsi) without affecting PWL the healthy (contralateral) paw. PBS injection did not affect PWL in either paw, n=6 mice/group. All data is expressed as mean±SEM. Abbreviations; PWT=paw withdrawal latency, CFA=complete Freuds Adjuvans.

### DETAILED DESCRIPTION

### Definitions

Non-peptide herein refers to a portion of the PICK1 inhibitor which does not comprise a peptide. A peptide is to be understood as comprising two or more α- or β-amino acids linked via amide bond(s). Thus non-peptide refers to a compound which does not comprise two or more α- or β-amino acids linked via amide bond(s). The non-peptide portion may comprise a single amino acid.

Lipophilic aliphatic group herein refers to an aliphatic group having lipophilic character. It may comprise an aliphatic chain or an aliphatic cycle. The term lipid as used herein refers to such lipophilic aliphatic group. The lipophilic aliphatic group may comprise a functional group, which may be used for attachment of the lipophilic aliphatic group to e.g. the NPEG linker to form the PICK1 inhibitor of the present disclosure. Example of lipophilic aliphatic groups include but are not limited to fatty acids, gonanes, sterols and steroids.

Bivalent herein refers to a compound comprising two sites for coordination, such as comprising two peptide ligands capable of coordinating to, such as binding to, a protein, such as PICK1. A bivalent peptide ligand, as referred to herein, may be a compound comprising two peptide ligands conjugated via a linker, such as to form a dimer of peptides. Thus, the bivalent peptide ligand as disclosed herein may also be referred to as a dimeric peptide ligand.

Functional group herein refers to a chemical group present in a chemical compound. A functional group comprises a reactivity, such as being nucleophile or electrophile and may be used for conjugating said chemical compound to other chemical compounds. Examples of functional groups include but are not limited to carboxylic acids, alcohols and amines.

Micellar structure or micelle herein refers to an arrangement of PICK1 inhibitors. As the term is used herein, a micelle has the arrangement in aqueous solution in which non-polar tails face inward and polar heads face outward (Example 3).

Radius of gyration (R_{g}) herein refers to the root mean square distance of the various particles of a body from the axis of rotation of said body. The radius of gyration is thus a measure of the size of said body.

Detectable moiety herein refers to a moiety which causes a detectable signal. Conventional moieties known to those of ordinary skill in the art for detection can be used such as a fluorophore, a chromophore, a radioisotope or an enzyme.

Amino acids, that are proteinogenic are named herein using either its 1-letter or 3-letter code according to the recommendations from IUPAC, see for example http://www.chem.qmw.ac.uk/iupac. If nothing else is specified, an amino acid may be of D or L-form. In a preferred embodiment, the amino acids of the present disclosure are L-amino acids.

α-carboxylic acid herein refers to the carboxylic acid conjugated to the α-carbon of an amino acid.

α-amine herein refers to the amine conjugated to the α-carbon of an α-amino acid.

β-amine herein refers to the amine conjugated to the β-carbon of a β-amino acid.

Ethylene glycol moiety, here refers to the structural unit that constitute a PEG or NPEG linker. A more technical name of a ‘ethylene glycol moiety’ is ‘oxyethylene’, and the chemical formula of the unit is here shown:

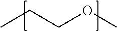

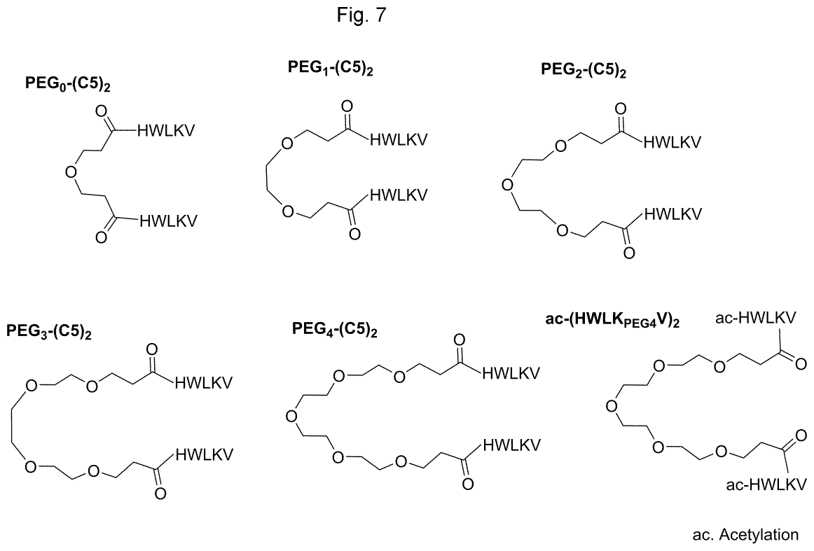

PEG, polyethylene glycol; PEG is a polymer of ethylene glycol_having the chemical formula C₂ₙ₊₂H₄ₙ₊₆Oₙ₊₂, and the repeating structure:

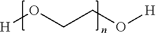

where PEGₓ refers to a PEG linker having x repeating ethylene glycol units, for example PEG₄, corresponds to a polymer of 4 ethylene glycol moieties (x=4). PEG₀ refers to a linker having x=0 and refers to a linker comprising two propionic acids covalently bound by an ether bond via the C3 carbons of each propionic acid moiety. Such structure is referred to herein as PEG₀ (FIG. 7) and has a structure according to Formula (III):

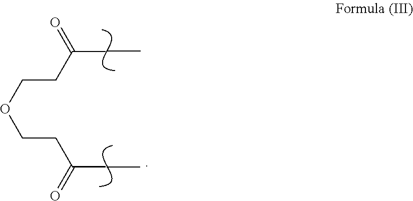

NPEG, is the linker type described herein, which is derived from the classical PEG linker, wherein one or more of the backbone oxygen atoms is replaced with a nitrogen atom. NPEGₓ refers to a NPEG linker having x repeating ethylene glycol units, for example NPEG₄, corresponds to a polymer of 4 ethylene glycol moieties (x=4), wherein one or more of the backbone oxygen atoms is replaced with a nitrogen atom. NPEG₀ refers to a linker having x=0 as defined above, wherein the ether is replaced by an amine.

PDZ, acronym combining the first letters of the first three proteins discovered to share the domain Postsynaptic density protein-95 (PSD-95), Drosophila homologue discs large tumor suppressor (DIgA), and Zonula occludens-1 protein (zo-1). PDZ domains are common structural domains of 80-90 amino-acids found in signaling proteins. Proteins containing PDZ domains often play a key role in anchoring receptor proteins in the membrane to cytoskeletal components.

Amide bond is formed by a reaction between a carboxylic acid and an amine (by concomitant elimination of water). Where the reaction is between two amino acid residues, the bond formed as a result of the reaction is known as a peptide linkage (peptide bond).

Ester bond is formed by a reaction between a carboxylic acid and an alcohol (by concomitant elimination of water).

Von Frey test, assess touch sensitivity with von Frey filaments. These filaments are applied to the underside of the paw after the mouse has settled into a comfortable position within a restricted area that has a perforated floor. The filaments are calibrated to flex when the set force is applied to the paw. Filaments are presented in order of increasing stiffness, until a paw withdrawal is detected.

Absent is to be understood as that the amino acid residues directly adjacent to the absent amino acid are directly linked to each other by a conventional amide bond.

AMPAR may also be referred to as AMPA receptor, AM PA-type glutamate receptor, or α-amino-3-hydroxy-5-methyl-4-isoxazolepropionic (AMPA) acid receptor is an ionotropic transmembrane receptor for glutamate that mediates fast synaptic transmission in the central nervous system (CNS). PICK1 interacts with AMPAR via the PDZ domain.

Chemical Structure of Bivalent PICK1 Inhibitor

The present disclosure provides PICK1 inhibitor which comprises a bivalent peptide ligand capable of binding to PICK1, the bivalent peptide ligand is further conjugated to a lipid. The lipid conjugated bivalent peptide ligand provide highly potent inhibitors of PICK, which may be used for treatment of diseases or disorders associated with maladaptive plasticity.

In one embodiment, a PICK1 inhibitor comprising a peptide portion and a non-peptide portion is provided, wherein the peptide portion consists of
a) a first peptide comprising an amino acid sequence of the general formula: X₁X₂X₃X₄X₅; and
b) a second peptide comprising an amino acid sequence of the general formula: X₁X₂X₃X₄X₅;

wherein
X₁ is H, N, F, or T, or is absent;
X₂ is W, S, E, or Y; or is absent;
X₃ is L, V, or I;
X₄ is K, I, or R; and
X₅ is V;

and wherein the non-peptide portion comprises:
c) a linker linking the first peptide to the second peptide, and
d) a lipophilic aliphatic group.

In one embodiment, a PICK1 inhibitor comprising a peptide portion and a non-peptide portion is provided, wherein the peptide portion consists of
a) a first peptide consisting of an amino acid sequence of the general formula: X₂X₃X₄X₅; and
b) a second peptide consisting of an amino acid sequence of the general formula: X₁X₂X₃X₄X₅;

wherein
X₁ is H, N, F, or T, or is absent;
X₂ is W, S, E, or Y; or is absent;
X₃ is L, V, or I;
X₄ is K, I, or R; and
X₅ is V;

and wherein the non-peptide portion comprises:
c) a linker linking the first peptide to the second peptide, and
d) a lipophilic aliphatic group.

In one embodiment, the PICK1 inhibitor has the generic structure of formula (I):

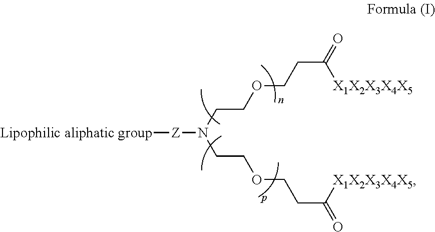

wherein
Z is a bond or a single amino acid;
n is an integer 0 to 12;
p is an integer 0 to 12.

Peptide Portion

The peptide ligand portion of the PICK1 inhibitor of the present disclosure provides binding of the PICK1 inhibitor to the PDZ-domain of PICK 1. The peptide portion of the PICK1 inhibitor of the present disclosure comprises a first and a second peptide. In one embodiment, the first and the second peptide are identical. In a separate embodiment, the first and the second peptides are different from each other. In one embodiment, the first and the second peptide are linked via a linker, such as to form a bivalent peptide ligand.

In one embodiment, the first and/or the second peptide is selected from the group consisting of HWLKV (SEQ ID NO: 54), FEIRV (SEQ ID NO: 34), NSIIV (SEQ ID NO: 5), NSVRV (SEQ ID NO: 8), NSLRV (SEQ ID NO: 53), NSIRV (SEQ ID NO: 6), NYIIV (SEQ ID NO: 13), NYIRV (SEQ ID NO: 14), TSIRV (SEQ ID NO: 18), YIIV (SEQ ID NO: 49), SVRV (SEQ ID NO: 44), EIRV (SEQ ID NO: 46), LRV, IIV, VRV, and IRV.

In one embodiment, the first and/or the second peptide is selected from the group consisting of HWLKV, NSVRV, NSLRV, NSIRV, TSIRV, EIRV, YIIV, IIV, VRV and IRV.

In one embodiment, the first and/or the second peptide is selected from the group consisting of NSVRV, NSLRV, NSIRV, TSIRV, EIRV, YIIV, IIV, VRV, and IRV.

In one embodiment, the first and/or the second peptide is selected from the group consisting of HWLKV, FEIRV, NSIIV, NSVRV, NSLRV, NSIRV, YIIV, SVRV, VRV, and LRV.

In one embodiment, the first and/or the second peptide is selected from the group consisting of FEIRV, NSIIV, NSVRV, NSLRV, NSIRV, YIIV, SVRV, VRV, and LRV.

In one embodiment, the first and/or second peptide is HWLKV, NSVRV or NSIRV.

In one embodiment, the first and/or the second peptide is HWLKV.

In one embodiment, the first and/or the second peptide comprises an amino acid sequence of the general formula: X₁X₂X₃X₄X₅, wherein:
X₁ is N, F, or T, or is absent;
X₂ is S, E, or Y; or is absent;
X₃ is V, L or I;
X₄ is I or R; and
X₅ is V.

In one embodiment, the first and/or the second peptide comprises an amino acid sequence of the general formula: X₁X₂X₃X₄X₅, wherein:
X₁ is N or T, or is absent;
X₂ is S, E, or Y; or is absent;
X₃ is V, L, or I;
X₄ is I or R; and
X₅ is V.

In one embodiment, the first and/or the second peptide comprises an amino acid sequence of the general formula: X₁X₂X₃X₄X₅, wherein:
X₁ is N or F, or is absent;
X₂ is S, E, or Y; or is absent;
X₃ is V, L, or I;
X₄ is I or R; and
X₅ is V.

In one embodiment, the first and/or second peptide comprises an amino acid sequence having a length in the range of 3 to 15 amino acids, such as in the range of 3 to 14 amino acid, for example in the range of 3 to 13 amino acids, such as in the range of 3 to 12 amino acid, for example in the range of 3 to 11 amino acids, such as in the range of 3 to 10 amino acid, for example in the range of 3 to 9 amino acids, such as in the range of 3 to 8 amino acid, for example in the range of 3 to 7 amino acids, such as in the range of 3 to 6 amino acid, for example in the range of 3 to 5 amino acids.

In a preferred embodiment, the first and/or second peptide comprises an amino acid sequence having a length in the range of 3 to 5 amino acids, such as having a length of 3 amino acids, such as having a length of 4 amino acids, such as having a length of 5 amino acids.

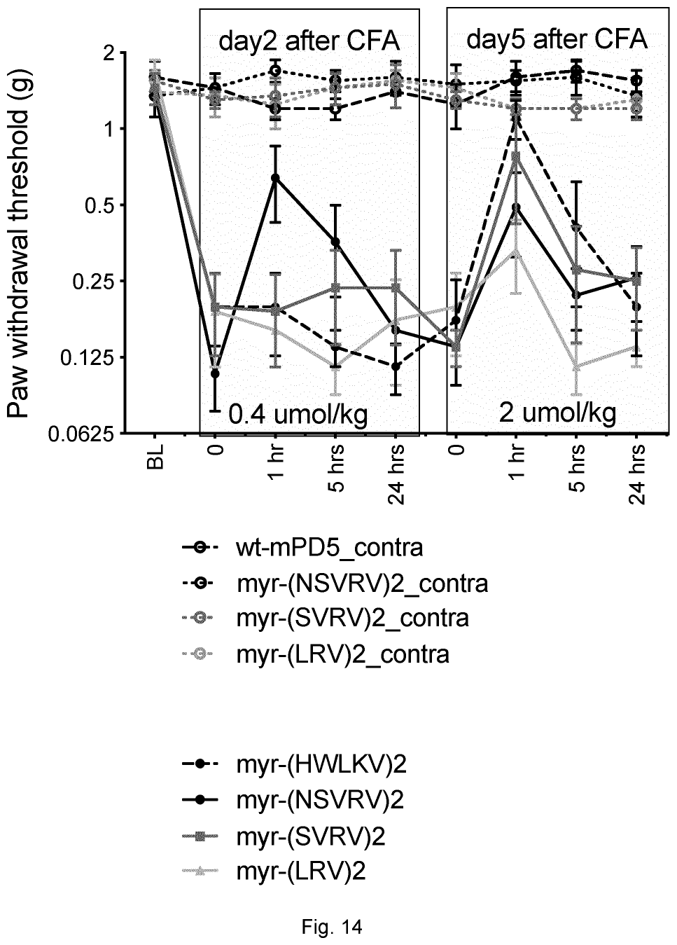

As demonstrated in Example 12 and FIG. 14 of the present disclosure, PICK1 inhibitors comprising a peptide portion consisting of a first and a second peptide having a length of 5, 4 or 3 amino acids demonstrate efficacy in alleviating pain, such alleviating inflammatory pain.

Similar or higher affinity towards PICK1 has previously been demonstrated for longer peptides comprising the sequence of the first and/or second peptides of the present disclosure (WO 2020/083905).

Non-Peptide Portion

The PICK1 inhibitor of the present disclosure comprises a non-peptide portion. The non-peptide portion of the PICK1 inhibitor comprises a linker which combines the first and the second peptides, such as to form a bivalent peptide ligand. The non-peptide portion further comprises a lipid which may be conjugated to said linker. In one embodiment, the lipid is directly linked to a nitrogen atom of the linker.

In a separate embodiment, the non-peptide portion further comprises a single amino acid. It is to be understood, as defined above, that the presence of a single amino acid in the non-peptide portion does not introduce a peptide into the non-peptide portion. As defined above, a peptide is to be understood as comprising two or more α- or β-amino acids linked via amide bonds. The presence of a single amino acid does not introduce such peptide as defined. In one embodiment, the single amino acid present in the non-peptide portion functions to provide a handle for attachment of a detectable moiety.

Linker

In one embodiment, the linker is an NPEG linker. The NPEG linker may comprise in the range of 0 to 24 ethylene glycol moieties wherein one or more of the backbone oxygen atoms is replaced with a nitrogen atom, such as in the range of 0 to 20, for example in the range of 0 to 16, such as in the range of 0 to 14, for example in the range of 0 to 12, for example in the range of 0 to 10, such as in the range of 0 to 8, for example in the range of 0 to 6, such as in the range of 0 to 4, for example in the range of 0 to 2 ethylene glycol moieties wherein one or more of the backbone oxygen atoms is replaced with a nitrogen atom. Preferably the NPEG-linker comprises 4 ethylene glycol moieties wherein one or more of the backbone oxygen atoms is replaced with a nitrogen atom. In one embodiment, the NPEG-linker comprises 3 ethylene glycol moieties wherein one or more of the backbone oxygen atoms is replaced with a nitrogen atom. In one embodiment, the NPEG-linker comprises 2 ethylene glycol moieties wherein one or more of the backbone oxygen atoms is replaced with a nitrogen atom. In one embodiment, the NPEG-linker comprises 1 ethylene glycol moieties wherein one or more of the backbone oxygen atoms is replaced with a nitrogen atom. In one embodiment, the NPEG-linker comprises 0 ethylene glycol moieties wherein one or more of the backbone oxygen atoms is replaced with a nitrogen atom, i.e. the linker has the structure of PEG₀ as disclosed in FIG. 7 with the oxygen atom replaced with a nitrogen. As defined above, an NPEG₀ linker refers to a linker having x=0 and refers to a linker comprising two propionic acid moieties combined in an amine bond via the C3 carbons of each propionic acid moiety. In one embodiment, the linker has a structure according to formula (III),

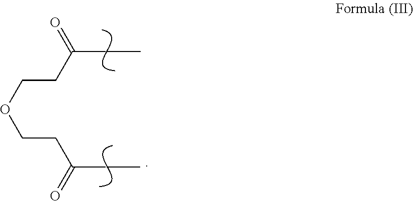

In one embodiment, the linker is an NPEG linker. The NPEG linker may comprise in the range of 1 to 24 ethylene glycol moieties wherein one or more of the backbone oxygen atoms is replaced with a nitrogen atom, such as in the range of 1 to 20, for example in the range of 1 to 16, such as in the range of 1 to 14, for example in the range of 1 to 12, for example in the range of 1 to 10, such as in the range of 1 to 8, for example in the range of 1 to 6, such as in the range of 1 to 4, for example in the range of 1 to 2 ethylene glycol moieties wherein one or more of the backbone oxygen atoms is replaced with a nitrogen atom.

The one or more nitrogen atom of the NPEG linker may be positioned at any position along the NPEG linker, such as for example positioned in the middle of the NPEG linker or positioned towards one end of the NPEG linker.

In one embodiment, one backbone oxygen of the NPEG-linker is replaced with a nitrogen atom.

The NPEG linker comprises functional groups in each end to provide for conjugation to the first and the second peptides. In one embodiment, the NPEG linker comprises a carboxylic acid in each end. The carboxylic acids of the NPEG linker may be bound to the N-termini of the first and the second peptides to provide conjugation of the linker to the first and the second peptide via amide bonds.

Lipophilic Aliphatic Group

The non-peptide portion of the PICK1 inhibitor of the present disclosure comprises a lipid. The lipid may be conjugated directly to the linker or may be conjugated to the linker via a single amino acid.

The lipids of the present disclosure are lipophilic aliphatic groups. The lipophilic aliphatic group present in the non-peptide portion of the PICK1 inhibitor of the present disclosure may be an aliphatic chain or an aliphatic cycle.

In one embodiment, the lipophilic aliphatic group is an aliphatic chain. The aliphatic chain may be a branched or unbranched chain. The aliphatic chain may be a saturated or unsaturated chain.

In one embodiment, the lipophilic aliphatic group is an aliphatic cycle. The aliphatic cycle may comprise a gonane structure, such as sterol. Alternatively, the aliphatic cycle may comprise a steroid, such as cholesterol. It is demonstrated in example 16 that a construct with cholesterol has activity in an animal model of pain. Preferably, the cholesterol moiety is linked to the N-PEG via an amino acid, such as for example asparagine for example betaAsp.

In one embodiment, the lipophilic aliphatic group present in the non-peptide portion of the PICK1 inhibitor of the present disclosure further comprises a functional group, such as a carboxylic acid, an alcohol or an amine. Said functional group provides conjugation of the lipophilic aliphatic group to the remainder of the PICK1 inhibitor, such as to the linker directly or to the linker via a single amino acid.

In one embodiment, the lipophilic aliphatic group comprises an alcohol.

In one embodiment, the lipophilic aliphatic group comprises a carboxylic acid.

In one embodiment, the lipophilic aliphatic group is an aliphatic chain comprising a carbocylic acid, thus being a fatty acid.

The lipophilic aliphatic group may be a C4-026 fatty acid. The lipophilic aliphatic group may be a saturated fatty acid or an unsaturated fatty acid. In one embodiment, the lipophilic aliphatic group is a 016 fatty acid or a 018 fatty acid.

In one embodiment, the lipophilic aliphatic group comprises in the range of 4 to 26 carbon atoms, such as in the range of 4 to 24, for example in the range of 4 to 22, such as in the range of 4 to 20, for example in the range of 4 to 18, such as in the range of 4 to 16, for example in the range of 4 to 14, such as in the range of 4 to 12, for example in the range of 4 to 10, such as in the range of 4 to 8, for example in the range of 4 to 6 carbon atoms.

In one embodiment, the lipophilic aliphatic group comprises in the range of 4 to 26 carbon atoms, such as in the range of 6 to 26, for example in the range of 8 to 26, such as in the range of 10 to 26, for example in the range of 12 to 26, such as in the range of 14 to 26, for example in the range of 16 to 26, such as in the range of 18 to 26, for example in the range of 20 to 26, such as in the range of 22 to 26, for example in the range of 24 to 26 carbon atoms.

In one embodiment, the lipophilic aliphatic group comprises in the range of 4 to 26 carbon atoms, such as in the range of 6 to 24, for example in the range of 8 to 22, such as in the range of 10 to 20, for example in the range of 12 to 18, such as in the range of 14 to 18, for example in the range of 14 to 16 carbon atoms or 16 to 18 carbon atoms.

In one embodiment, the lipophilic aliphatic group is selected from the group consisting of acetic acid, butyric acid, caproic acid, caprylic acid, capric acid, lauric acid, myristic acid, palmitic acid, margaric acid, stearic acid, arachidic acid, behenic acid, lignoceric acid, cerotic acid, caproleic acid, lauroleic acid, myristoleic acid, palmitoleic acid, oleic acid, linoleic acid, linolenic acid, gadoleic acid, erucic acid. In a preferred embodiment, the lipophilic aliphatic group is selected from the group consisting of capric acid, lauric acid, myristic acid, palmitic acid, margaric acid, stearic acid. In a more preferred embodiment, the lipophilic aliphatic group is myristic acid.

In embodiments where the lipophilic aliphatic group is a fatty acid moiety, it may be saturated or unsaturated in cis or trans configuration. Example 16 demonstrates that for a C14 fatty acid moiety, unsaturated (cis or trans) and saturated moieties have similar effects in vivo. In one embodiment, the fatty acid moiety is polyunsaturated such as having one, two or three double bonds. In some embodiments, one double bond is in the n-3 or n-6 position.

Fatty acid moieties may be modified by having an additional functional group in the end opposite the carboxylic acid group. Such functional group may be an additional carboxylic acid, an alcohol, a ketone or an aldehyde. Example 16 demonstrates that a lipophilic aliphatic group may include a terminal carboxylic acid and have efficacy in an animal model of pain.

In one embodiment, the lipophilic aliphatic group is myristic acid, also referred to herein as myristoyl or myr.

In one embodiment, the lipophilic aliphatic group is selected from the group consisting of myristic acid, palmitic acid, stearic acid, Docosahexaenoic acid (DHA), and cis-9-Octadecenoic acid.

In one embodiment, the lipophilic aliphatic group is a diacid, such as for example tetradecanedioic acid, hexadecanedioic acid, or octadecanedioic acid.

Optional Amino Acid

The non-peptide portion may further comprise a single amino acid. Such single amino acid present in the non-peptide portion may function to provide a handle for attachment of a the lipophilic aliphatic group or for attachment of a detectable moiety.

In one embodiment, the one amino acid is an α-amino acid or a β-amino acid. The one amino acid may be selected from the group consisting of Asp, β-Asp, β-Ser (3-Amino-2-(hydroxymethyl)propanoic acid), β-homo-Ser (3-Amino-4-hydroxybutyric acid) and β-Lys (3,6-Diaminohexanoic acid).

As shown by the inventors in example 11, the lipophilic aliphatic group is essential for the invention to have a functional effect in vivo.

Connectivity

In one embodiment, the peptide and the non-peptide portions of the PICK1 inhibitor of the present disclosure are conjugated to form a PICK1 inhibitor having the generic structure of Formula (I).

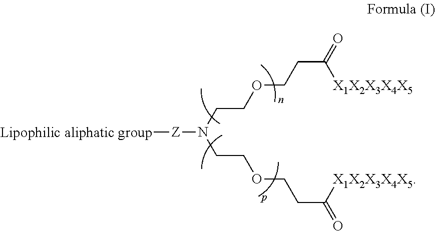

It is to be understood that when for example the lipophilic aliphatic group is described as being a fatty acid, only the carbonyl group of the carboxylic acid of the fatty acid is present in the PICK1 inhibitor. Upon conjugation of the fatty acid to e.g. an amine of the linker, an amide bond is formed with concomitant loss of water. Hence, the different components which are combined to form the PICK1 inhibitor of the present disclosure may arise from the described compounds. In the specific case of the lipophilic aliphatic group being a diacid, in one embodiment, only one of the carboxylic acids is reacted to form an amide with the amine of the linker, while the second carboxylic acid remains a carboxylic acid in the final PICK1 inhibitor.

The NPEG linker of present disclosure may for example comprise a carboxylic acid in each end. It is to be understood that the resulting NPEG linker found in the PICK1 inhibitor does not comprise the carboxylic acids but only the carbonyl groups which are present in the amide bonds formed when conjugating the NPEG linker to the first and/or the second peptide. Thus, in one embodiment, the NPEG linker is conjugated to the first and/or the second peptide via an amide bond formed between the carboxylic acids of the NPEG linker and the N-terminus of the first and/or second peptides. In one embodiment, the NPEG linker is conjugated to the first and/or the second peptide via an amide bond formed between the carboxylic acids of the NPEG linker and a side chain functional group of an amino acid in the first and/or the second peptide, such as by formation of an amide bond between the carboxylic acid of the NPEG linker and an amine of a lysine sidechain in the first and/or the second peptide to form an amide.

The nitrogen atom of the NPEG linker may be further conjugated to the lipophilic aliphatic group either directly or via a single amino acid. Thus in one embodiment, the lipophilic aliphatic group is conjugated via a functional group, such as a carboxylic acid, to the nitrogen atom of the NPEG linker, such as by forming an amide.

In one embodiment, a single amino acid is conjugated to the nitrogen atom of the NPEG linker via the α-carboxylic acid to form an amide and is further conjugated to the lipophilic aliphatic group. The further conjugation to the lipophilic aliphatic group may be via the α- or β-amine (α- or β-amino acid, respectively) to form an amide bond or via a side chain functional group, such as a carboxylic acid, an alcohol or an amine to form an amide bond or an ester bond.

Preferred Structures

In one embodiment the PICK1 inhibitor has a structure according to formula (II)

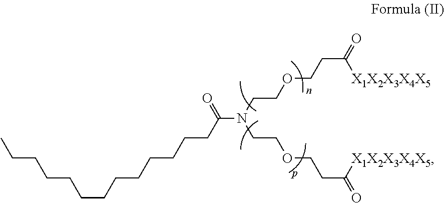

wherein
n is an integer 0 to 12, preferably 2;
p is an integer 0 to 12, preferably 2.

In a preferred embodiment, the PICK1 inhibitor has a structure according to formula (II), wherein n is 2 and p is 2. Such structure is referred to herein as myr-NPEG₄-(HWLKV)₂.

Other preferred structures are disclosed below. The unsaturation may be in trans or cis configuration.

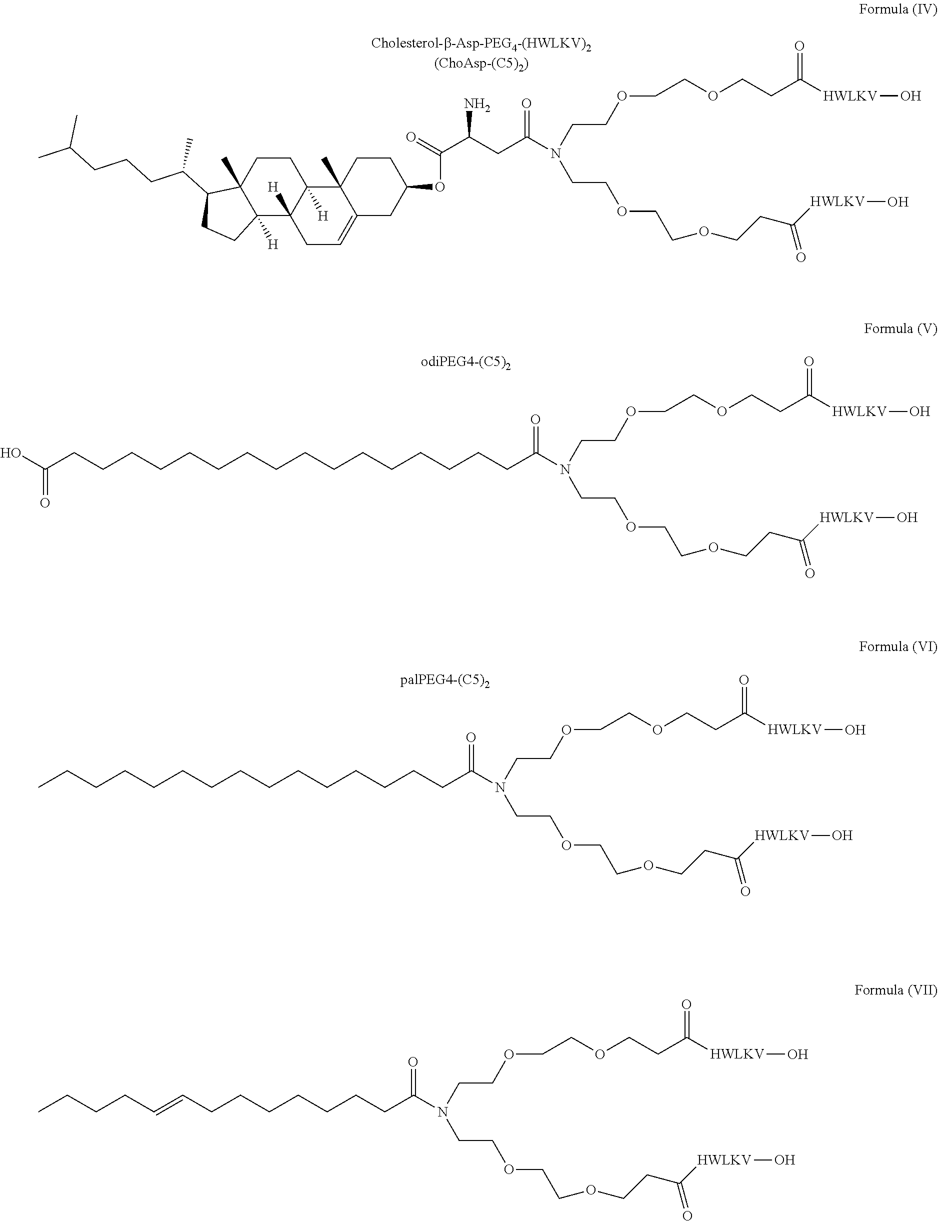

Mechanism of Action

As demonstrated in the present disclosure, example 3, it has been surprisingly found that the PICK1 inhibitor of the present invention is capable of forming micellar structures. It is hypothesized that the improved potency of the PICK1 inhibitor of the present invention, as compared to the bivalent peptide ligand not comprising a lipid and therefore not capable of forming micellar structures, is due to formation of this micellar structure.

PICK1 is known to be present in a dimer conformation, with dimerization mediated by the BAR domain. It has been reported that dimerization of the dimeric PICK1, providing dimers of dimers, such as tetramers, results in auto-inhibition of the protein function (Karlsen, M. L. et al. 2015). It can thus be hypothesized that the micellar PICK1 inhibitor is capable of binding and bringing together several PICK1 proteins, thereby leading to the observed effective inhibition of PICK1.

Thus, in one embodiment, the PICK1 inhibitor self-assembles into a higher order structure in solution, such as self-assemble to form micellar structures.

In one embodiment the higher order structure has a radius of gyration (Rg) of at least 15 Å, such as at least 17 Å, for example at least 19 Å, such as at least 20 Å, for example at least 21 Å, such as at least 22 Å, for example at least 23 Å.

In one embodiment the higher order structure has a radius of gyration (Rg) of at least 15 Å, such as at least 17 Å, for example at least 19 Å, such as at least 20 Å, for example at least 21 Å, such as at least 22 Å, for example at least 23 Å, such as at least 24 Å, for example at least 25 Å, such as at least 26 Å, for example at least 27 Å, such as at least 28 Å, for example at least 29 Å, such as at least 30 Å, for example at least 31 Å

In one embodiment, the higher order structures are formed from in the range of 4 to 20 PICK1 inhibitors, such as in the range of 4 to 18, for example in the range of 4 to 16, such as in the range of 4 to 14, for example in the range of 4 to 12, such as in the range of 4 to 10, for example in the range of 4 to 8, such as in the range of 6 to 8 PICK1 inhibitors.

In one embodiment, the higher order structures are formed from in the range of 4 to 40 PICK1 inhibitors, such as in the range of 6 to 40, for example in the range of 8 to 40, such as in the range of 10 to 40, for example in the range of 12 to 40, such as in the range of 14 to 40, for example in the range of 16 to 40, such as in the range of 18 to 40, for example in the range of 20 to 40, such as in the range of 22 to 40, for example in the range of 24 to 40, such as in the range of 26 to 40, for example in the range of 28 to 40, such as in the range of 30 to 40, for example in the range of 32 to 40, such as in the range of 34 to 40, for example in the range of 36 to 40, such as in the range of 38 to 40 PICK1 inhibitors.

In one embodiment, the higher order structures are formed from in the range of 4 to 40 PICK1 inhibitors, such as in the range of 4 to 38, for example in the range of 4 to 36, such as in the range of 4 to 34, for example in the range of 4 to 32, such as in the range of 4 to 30, for example in the range of 4 to 28, such as in the range of 4 to 26, for example in the range of 4 to 24, such as in the range of 4 to 22, for example in the range of 4 to 20, such as in the range of 4 to 18, for example in the range of 4 to 16, such as in the range of 4 to 14, for example in the range of 4 to 12, such as in the range of 4 to 10, for example in the range of 4 to 8, such as in the range of 4 to 6 PICK1 inhibitors.

In one embodiment, the higher order structures are formed from in the range of 4 to 40 PICK1 inhibitors, such as in the range of 6 to 38, for example in the range of 6 to 36, such as in the range of 8 to 34, for example in the range of 8 to 32, such as in the range of 10 to 30, for example in the range of 10 to 28, such as in the range of 12 to 26, for example in the range of 14 to 24, such as in the range of 16 to 24, for example in the range of 16 to 22, such as in the range of 18 to 22, for example in the range of 19 to 21, such as 20 PICK1 inhibitors.

In one embodiment, a micelle is provided comprising a PICK1 inhibitor as disclosed herein.

In one embodiment, a micelle is provided comprising a PICK1 inhibitor comprising
a) a first peptide comprising an amino acid sequence of the general formula: X₁X₂X₃X₄X₅; and
b) a second peptide comprising an amino acid sequence of the general formula: X₁X₂X₃X₄X₅;

wherein:
X₁ is H, N, F, or T, or is absent;
X₂ is W, S, E, or Y; or is absent;
X₃ is L, V, or I;
X₄ is K, I, or R; and
X₅ is V;
c) a linker linking the first peptide to the second peptide, and
d) a lipophilic aliphatic group.

As demonstrated herein, the PICK1 inhibitor of the present disclosure is capable of binding to the PDZ domain of PICK1.

In the native PICK1 dimer conformation, the distance between the PDZ domains of the PICK1 dimer is estimated to be ˜180 Å. In the PICK1 inhibitor comprising an NPEG linker having a length of 4 ethylene glycol moieties, the distance between the first and the second peptide is estimated to span ˜43 Å. Such PICK1 inhibitor will therefore not be able to bind the two PDZ domains found in a single PICK1 dimer. This supports the hypothesis that a single PICK1 inhibitor as disclosed herein will function by binding and bringing together two PICK1 dimers, leading to inhibition of PICK1. It follows that a micellar structure formed by several PICK1 inhibitor as disclosed herein, is likely to be able to bind and bring together two or more dimers of PICK1, thereby leading to the effective inhibition of PICK1 as disclosed herein.

Thus, in one embodiment, the PICK1 inhibitor or the micellar structure as disclosed herein binds to the PDZ domain of two or more PICK1 proteins, leading to inhibition of PICK1. In one embodiment, the two or more PICK1 proteins bound by the PICK1 inhibitor of the present disclosure are present in two or more dimers of PICK1.

In one embodiment, binding of the PICK1 inhibitor to PICK1 results in formation of higher oligomeric states of PICK1, such as trimers, tetramers, pentamers, hexamers, heptamers or octamers of PICK1. In one embodiment, binding of the PICK1 inhibitor to PICK1 result in formation of tetramers, hexamers or octamers of PICK1.

In one embodiment, the PICK1 inhibitor of the present disclosure brings together two or more PICK1 proteins. In one embodiment, the compound brings together four PICK1 proteins, such as five PICK1 proteins, for example six PICK1 proteins, such as seven PICK1 proteins, for example eight PICK1 proteins, such as nine PICK1 proteins, for example 10 PICK1 proteins.

Inhibition of PICK1 by binding to the PICK1 inhibitor of the present disclosure may result in the PICK1 protein no longer being capable of interacting with AMPAR, thereby preventing PICK1 in controlling trafficking of AMPAR. Thus, in one embodiment, the PICK1 inhibitor is capable of inhibiting a protein-protein interaction between PICK1 and AMPAR. This may thus prevent PICK1 from down-regulating GluA2 and prevent CP-AMPARs formation thereby preventing a maladaptive type of plasticity in response to abnormal levels of glutamate in the synapse. This in turn can prevent for example neuropathic pain and cocaine addiction.

The PICK1 inhibitor of the present disclosure possesses high affinity towards the PDZ domain of PICK1. In one embodiment, the PICK1 inhibitor of the present disclosure has a Ki for PICK1 inferior to 10 nM, such as inferior to 9 nM, such as inferior to 8 nM, such as inferior to 7 nM, such as inferior to 6 nM, such as inferior to 5 nM, such as inferior to 4 nM, such as inferior to 3 nM, such as inferior to 2 nM, such as inferior to 1 nM, such as inferior to 0.5 nM.

The affinity of the PICK1 inhibitor of the present disclosure towards the PDZ domain of PICK1 may be determined by fluorescent polarization (FP) as described herein, example 4.

The ability of the PICK1 inhibitor of the present disclosure of forming higher order structures may be determined by size exclusion chromatography (SEC) or Small-angle X-ray scattering (SAXS) as described herein, example 3.

Detectable Moiety

In one embodiment, the PICK1 inhibitor of the present disclosure further comprises a detectable moiety. Conventional moieties known to those of ordinary skill in the art for detection can be used such as a fluorophore, a chromophore, a radiosotope or an enzyme. The presence of a detectable moiety in the PICK1 inhibitor allows for labelling and visualization of PICK1 upon binding to the PICK1 inhibitor.

In one embodiment, the detectable moiety is conjugated to the first and/or the second peptide. In one embodiment, the detectable moiety is conjugated to the single amino acid of the non-peptide portion.

In one embodiment, the detectable moiety is a fluorophore, such as 5, 6-carboxyltetramethylrhodamine (TAMRA) or indodicarbocyanine (Cy5).

In another embodiment, the detectable moiety comprises or consists of a radioisotope.

The radioisotope may be selected from the group consisting of ¹²⁵I, ^{99m}Tc, ¹¹¹In, ⁶⁷Ga, ⁸⁸Ga, ⁷²As, ⁸⁹Zr, ¹²³I, ¹⁸F and ²⁰¹Tl.

Diseases and Disorders

The present invention provides a pharmaceutical composition for treatment of diseases and/or disorders associated with maladaptive plasticity. In one embodiment, a pharmaceutical composition comprising a PICK1 inhibitor as disclosed herein or a micelle as disclosed herein is provided. The pharmaceutical composition may comprise the PICK1 inhibitor or the micelle of the present disclosure in a pharmaceutically accepted carrier.

AM PA-type glutamate receptors (AMPARs) are, in contrast to NMDA-type glutamate receptors (NMDARs), usually only permeable to monovalent cations (i.e. Na⁺ and K⁺) due to presence of GluA2 subunits in the tetrameric receptor complex. Plasticity changes in response to a strong and sustained depolarization, however, result in a switch to AMPARs with increased conductance and Ca²⁺ permeability (CP-AMPARs) in several types of synapses and this switch renders the synapse hypersensitive. Mechanistically, expression of CP-AMPARs involves an initial PICK1-dependent down-regulation of GluA2 containing AMPARs, which is mediated by the interaction between the PICK1 PDZ domain and the C-terminus of the GluA2 subunit of the AMPARs. This in turn allows for insertion of GluA2 lacking receptors in the synapse (Slot hypothesis) rendering the synapse Ca²⁺-permeable and hypersensitive.

CP-AMPARs are critically involved in the mediating craving after withdrawal from cocaine self-administration in rats (Conrad et al 2008). PICK1 has been implicated in the expression of CP-AMPAR in the VTA dopaminergic neurons in midbrain and in nucleus accumbens during development of cocaine craving (Luscher et al 2011 and Wolf et al 2010) suggesting PICK1 as a target in cocaine addiction. A CPP-conjugated bivalent peptide inhibitor of PICK1 has been reported to dose-dependently attenuate the reinstatement of cocaine seeking in rats (Turner et al. 2020). Thus in one embodiment, administration of the PICK1 inhibitor of the present disclosure reduces cocaine craving in drug addiction, such as cocaine addiction.

Upregulation of AMPA-type glutamate receptors (AMPARs) in the dorsal horn (DH) neurons causes central sensitization, a specific form of synaptic plasticity in the DH sustainable for a long period of time (Woolf et al 2000 and Ji et al 2003). Moreover, both peripheral inflammatory pain and nerve injury induced pain, cause upregulation of Ca²⁺-permeable AMPARs (CP-AMPARs) (Vikman et al 2008, Gangadharan et al 2011 and Chen et al 2013). Initial evidence for a role of PICK1 in neuropathic pain came from Garry et al 2003 demonstrating that peptide inhibitors of PICK1 alleviated pain induced by chronic constriction injury (CCI). Subsequently, it was demonstrated the shRNA mediated knock down of PICK1 alleviated complete Freud's adjuvans (CFA) induced inflammatory pain and it was found that PICK1 knock-out mice completely fail to develop pain in response to spinal nerve ligation (SNL) (Wang et al 2011 and Atianjoh et al 2010). Indeed, administration of the PICK1 inhibitor of the present disclosure reduces mechanical allodynia in a model of neuropathic pain (SNI model—example 8), inflammatory pain (CFA model—examples 7 and 16), and thermal (heat) allodynia in a model of inflammatory pain (CFA model—example 17). Therefore, in one embodiment the pain is mechanical or thermal allodynia or hyperalgesia. In another embodiment the pain is inflammatory pain

Both TAR DNA-binding protein 43 (TDP-43) pathology and failure of RNA editing of the AMPA receptor subunit GluA2, are etiology-linked molecular abnormalities that concomitantly occur in the motor neurons of the majority of patients with amyotrophic lateral sclerosis (ALS). Pain symptoms in a mouse model with conditional knock-out of the RNA editing enzyme adenosine deaminase acting on RNA 2 (ADAR2) are relieved by the AMPAR antagonist perampanel, suggesting a likely symptomatic relief by the PICK1 inhibitor of the present disclosure.

Given the effect of the compounds of the present disclosure on pain and addiction, it is reasonable to expect also good efficacy on patient with comorbidity e.g. pain patients also suffering from addiction.

Similar central sensitization is thought to underlie the allodynia in hyperalgesic priming, which serves as an experimental model for lower back pain and migraine (Kandasamy et al 2015).

Similarly, the etiology for tinnitus holds several parallels with neuropathic pain including central sensitization (Vanneste et al 2019, Peker et al 2016, and Moller et al 2007).

A role for PICK1 in the surface stabilization/insertion of CP-AMPARs has been described for oxygen-glucose depletion in cultured hippocampal neurons (Clem et al 2010 and Dixon et al 2009). This evokes PICK1 as a putative target in the protection of neural death after ischemic insult.

Loss of PICK1 has been demonstrated to protect neurons in vitro and in vivo against spine loss in response to amyloid beta (Marcotte et al 2018 and Alfonso et al 2014). Consequently, PICK1 is a putative target for symptomatic and perhaps preventive treatment of Alzheimer's disease.

PICK1 interacts and inhibits the E3 ubiquitin ligase Parkin, which is involved in mitophagy. Parkin loss of function is associated with both sporadic and familial Parkinson's disease (PD). As a result, PICK1 KO mice are resistant to 1-methyl-4-phenyl-1,2,3,6-tetrahydropyridine (MPTP)-mediated toxicity (He et al 2018).

Consequently, PICK1 is a putative target for symptomatic and perhaps preventive treatment of Parkinson's disease.

Overstimulation of glutamate receptors resulting in excessive intracellular calcium concentrations is a major cause of neuronal cell death in epilepsy. The GluR2 (GluA2) hypothesis states that following a neurological insult such as an epileptic seizure, the AMPA receptor subunit GluR2 protein is downregulated. This increases the likelihood of the formation of GluR2-lacking, calcium-permeable AMPA receptor which might further enhance the toxicity of the neurotransmitter, glutamate (Lorgen et al 2017).

PICK1 is overexpressed in tumor cells as compared to adjacent normal epithelia in breast, lung, gastric, colorectal, and ovarian cancer. As judged by immunostaining breast cancer tissue microarrays, high levels of PICK1 expression correlates with shortened span of overall survival. Accordingly, transfection of MDA-MB-231 cells with PICK1 siRNA decreased cell proliferation and colony formation in vitro and inhibited tumorigenicity in nude mice (Zhang et al 2010). Consequently, PICK1 is a putative target for cancer treatment and prognostics.

In one embodiment, a PICK1 inhibitor, a micelle or a pharmaceutical composition as disclosed herein is provided for use as a medicament.

In one embodiment, a PICK1 inhibitor, a micelle or a pharmaceutical composition as disclosed herein is provided for use in the prophylaxis and/or treatment of a disease or disorder associated with maladaptive plasticity.

In one embodiment, a method of providing prophylaxis and/or treatment of a disease or disorder associated with maladaptive plasticity in a subject is provided, the method comprising administering the PICK1 inhibitor, the micelle or the pharmaceutical composition of the present disclosure to the subject.

In one embodiment, use of the PICK1 inhibitor, the micelle or the pharmaceutical composition of the present disclosure is provided for the manufacture of a medicament for the treatment of diseases and/or disorders associated with maladaptive plasticity.

In one embodiment, the disease or disorder associated with maladaptive plasticity is pain, drug addiction, amyotrophic lateral sclerosis, epilepsy, tinnitus, migraine, cancer, ischemia, Alzheimer's disease, and/or Parkinson's disease.

In one embodiment, the disease or disorder associated with maladaptive plasticity is pain, such as neuropathic pain. The pain can be inflammatory pain or neuropathic pain. The pain, to be treated, may be chronic pain, which may be chronic neuropathic pain or chronic inflammatory pain. The neuropathic pain may be induced by damage to the peripheral or central nervous system as a result of traumatic injury, surgery, or diseases such as diabetes or autoimmune disorders. The neuropathic pain may be induced by treatment with chemotherapy. Where pain persists, the condition is chronic neuropathic pain. Chronic inflammatory pain may be induced by inflammation after nerve injury, as well as being initiated by inflammation induced by alien matter, where mediators released by immune cells cause a sensitization of pain pathways, i.e. a ‘wind up’ of sensory neurons located in the spinal cord. Thus, an effective analgesic drug must be able to reach spinal cord tissue and find its target, in this case PICK1, in order to have a pain-relieving effect. Thereby, the compounds must be able to pass the blood-brain barrier and/or blood-spinal cord barrier to be able to reach spinal cord tissue.

In one embodiment, the disease or disorder associated with maladaptive plasticity is drug addiction, such as cocaine addiction, opioid addiction, or morphine addiction.

In one embodiment, the disease or disorder associated with maladaptive plasticity is cancer such as breast cancer, for example histological grade, lymph node metastasis, Her-2/neu-positivity, and triple-negative basal-like breast cancer.

In one embodiment, the disease or disorder associated with maladaptive plasticity is amyotrophic lateral sclerosis.

In one embodiment, the disease or disorder associated with maladaptive plasticity is epilepsy.

In one embodiment, the disease or disorder associated with maladaptive plasticity is tinnitus.

In one embodiment, the disease or disorder associated with maladaptive plasticity is migraine.

In one embodiment, the disease or disorder associated with maladaptive plasticity is stroke or ischemia.

In one embodiment, the disease or disorder associated with maladaptive plasticity is Alzheimer's disease.

In one embodiment, the disease or disorder associated with maladaptive plasticity is Parkinson's disease.

In yet another embodiment, the compound as disclosed herein is for use in the prophylaxis and/or treatment of head injury.

In yet another embodiment, the compound as disclosed herein is for use in the prophylaxis and/or treatment and/or diagnosis of cancer, such as breast cancer.

Subjects at risk or presently suffering from the above disorders and diseases may be given either prophylactic treatment to reduce the risk of the disorder or disease onset or therapeutic treatment following the disorder or disease onset. The subject may be a mammalian or human patient.

Administration

Conventional methods, known to those of ordinary skill in the art of medicine, can be used to administer the PICK1 inhibitor of the present disclosure to the patient. The PICK1 inhibitor of the present disclosure can be administered alone, or in combination with other therapeutic agents or interventions.

In one embodiment, the PICK1 inhibitor of the present disclosure is administered by parenteral administration, such as intravenous, intraperitoneal, intramuscular, intrathecal, transcutaneous, transmucosal, or subcutaneous administration. In one embodiment, the PICK1 inhibitor of the present disclosure is administered by intrathecal or subcutaneous administration. In a preferred embodiment, the PICK1 inhibitor of the present disclosure is administered by subcutaneous administration. As demonstrated in example 15, the PICK1 inhibitor of the present disclosure possesses a high solubility rendering it suitable for such subcutaneous administration at an effective dose.

Diagnosis

The PICK1 inhibitor of the present disclosure may comprise a detectable moiety. Such PICK1 inhibitor may thus be used for diagnosis, such as by detecting PICK1 in a tissue or a sample.

Thus, the present disclosure provides a PICK1 inhibitor as disclosed herein for use in diagnosis of a disease or disorder associated with maladaptive plasticity.

PICK1 is overexpressed in tumor cells as compared to adjacent normal epithelia in breast, lung, gastric, colorectal, and ovarian cancer. As judged by immunostaining breast cancer tissue microarrays, high levels of PICK1 expression correlates with shortened span of overall survival. Accordingly, transfection of MDA-MB-231 cells with PICK1 siRNA decreased cell proliferation and colony formation in vitro and inhibited tumorigenicity in nude mice (Zhang et al 2010). Consequently, PICK1 is a putative target for cancer treatment and prognostics.

In one embodiment, the PICK1 inhibitor as disclosed herein is for use in diagnosis of a disease or disorder associated with maladaptive plasticity is cancer, such as breast cancer. In one embodiment, the breast cancer is selected from histological grade, lymph node metastasis, Her-2/neu-positivity, and triple-negative basal-like breast cancer.

The present disclosure further provides a method of diagnosing breast cancer in a subject in need thereof, the method comprising the steps of:
a. obtaining a tissue sample from said subject;
b. staining the sample with the compound as disclosed herein;
c. determining the level of PICK1 in the sample; and
d. comparing the level of PICK1 in the sample to a healthy standard,

wherein an increased level of PICK1 in the sample is indicative of said individual having breast cancer.

The present disclosure further provides a method for predicting the prognosis for a subject suffering from breast cancer, the method comprising the steps of:
a. obtaining a tissue sample from said subject;
b. staining the sample with the compound as disclosed herein;
c. determining the level of PICK1 in the sample; and
d. comparing the level of PICK1 in the sample to a healthy standard,

wherein an increased level of PICK1 in the sample is indicative of poor prognosis.

In one embodiment, the PICK1 inhibitor as disclosed herein is used in stratification of subjects suffering from a disease associated with maladaptive plasticity into responders and non-responders of treatment with said PICK1 inhibitor. Such stratification may be used for assessing efficacy of PICK1 inhibitors having a bivalent or multivalent interaction with PICK1 prior to initializing other methods of treatment, such as AAV based therapies resulting in similar mechanisms of treatment, such as PICK1 inhibition. Advantages of such stratification include that only responders to the mechanism of treatment, such as PICK1 inhibition, will receive the long-lasting irreversible treatment of AAV based therapies. AAV based therapies are described in co-pending applications (PCT/EP2019/078736 and EP20161524.2).

Thus in one embodiment, the PICK1 inhibitor of the present disclosure is used for stratifying patients with a disease and/or disorder associated with maladaptive plasticity into predictable treatment responders of the gene therapy.

In one embodiment, the PICK1 inhibitor of the present disclosure is used in stratification of subjects suffering from a disease associated with maladaptive plasticity into responders and non-responders of treatment with said compound.

Items
1. A PICK1 inhibitor comprising a peptide portion and a non-peptide portion, wherein the peptide portion consists of
   a) a first peptide comprising an amino acid sequence of the general formula: X₁X₂X₃X₄X₅; and
   b) a second peptide comprising an amino acid sequence of the general formula: X₁X₂X₃X₄X₅;
   wherein
   X₁ is H, N, F, or T, or is absent;
   X₂ is W, S, E, or Y; or is absent;
   X₃ is L, V, or I;
   X₄ is K, I, or R; and
   X₅ is V;
   and wherein the non-peptide portion comprises:
   c) a linker linking the first peptide to the second peptide, and
   d) a lipophilic aliphatic group.
2. The PICK1 inhibitor according to any one of the preceding items, wherein the first and/or the second peptide is selected from the group consisting of HWLKV, FEIRV, NSIIV, NSVRV, NSLRV, NSIRV, NYIIV, NYIRV, TSIRV, YIIV, SVRV, EIRV, LRV, IIV, VRV, and IRV.
3. The PICK1 inhibitor according to any one of the preceding items, wherein the first and/or the second peptide is selected from the group consisting of HWLKV, NSVRV, NSLRV, NSIRV, TSIRV, EIRV, YIIV, IIV, VRV and IRV.
4. The PICK1 inhibitor according to any one of the preceding items, wherein the first and/or the second peptide is selected from the group consisting of NSVRV, NSLRV, NSIRV, TSIRV, EIRV, YIIV, IIV, VRV, and IRV.
5. The PICK1 inhibitor according to any one of the preceding items, wherein the first and/or the second peptide is selected from the group consisting of HWLKV, FEIRV, NSIIV, NSVRV, NSLRV, NSIRV, YIIV, SVRV, VRV, and LRV.
6. The PICK1 inhibitor according to any one of the preceding items, wherein the first and/or the second peptide is selected from the group consisting of FEIRV, NSIIV, NSVRV, NSLRV, NSIRV, YIIV, SVRV, VRV, and LRV.
7. The PICK1 inhibitor according to any one of the preceding items, wherein the first and/or the second peptide is HWLKV.
8. The PICK1 inhibitor according to item 1, wherein:
   X₁ is N, F, or T, or is absent;
   X₂ is S, E, or Y; or is absent;
   X₃ is V, L or I;
   X₄ is I or R; and
   X₅ is V.
9. The PICK1 inhibitor according to item 1, wherein:
   X₁ is N or T, or is absent;
   X₂ is S, E, or Y; or is absent;
   X₃ is V, L or I;
   X₄ is I or R; and
   X₅ is V.
10. The PICK1 inhibitor according to item 1, wherein:
   X₁ is N or F, or is absent;
   X₂ is S, E, or Y; or is absent;
   X₃ is V, L or I;
   X₄ is I or R; and
   X₅ is V.
11. The PICK1 inhibitor according to any one of the preceding items, wherein the first peptide is identical to the second peptide.
12. The PICK1 inhibitor according any one of the preceding items, wherein the linker is an NPEG linker.
13. The PICK1 inhibitor according to any one of the preceding items, wherein the NPEG linker comprises in the range of 0 to 24 ethylene glycol moieties wherein one or more of the backbone oxygen atoms is replaced with a nitrogen atom, such as in the range of 0 to 20, for example in the range of 0 to 16, such as in the range of 0 to 14, for example in the range of 0 to 12, for example in the range of 0 to 10, such as in the range of 0 to 8, for example in the range of 0 to 6, such as in the range of 0 to 4, for example in the range of 0 to 2 ethylene glycol moieties wherein one or more of the backbone oxygen atoms is replaced with a nitrogen atom, preferably the NPEG-linker comprises 4 ethylene glycol moieties wherein one or more of the backbone oxygen atoms is replaced with a nitrogen atom.
14. The PICK1 inhibitor according to any one of the preceding items, wherein one backbone oxygen of the NPEG-linker is replaced with a nitrogen atom.
15. The PICK1 inhibitor according to any one of the preceding items, wherein the NPEG linker comprises a carboxylic acid in each end.
16. The PICK1 inhibitor according to any one of the preceding items, wherein the one or more nitrogen atom of the NPEG linker is positioned at any position along the NPEG linker, such as for example positioned in the middle of the NPEG linker or positioned towards one end of the NPEG linker.
17. The PICK1 inhibitor according to any one of the preceding items, wherein the NPEG linker is conjugated to the first and/or the second peptide via an amide bond formed between the carboxylic acids of the NPEG linker and the N-terminus of the first and/or second peptides.
18. The PICK1 inhibitor according to any one of the preceding items, wherein the lipophilic aliphatic group is an aliphatic chain or an aliphatic cycle.
19. The PICK1 inhibitor according to any one of the preceding items, wherein the lipophilic aliphatic group is an aliphatic branched or unbranched chain.
20. The PICK1 inhibitor according to any one of the preceding items, wherein the lipophilic aliphatic group is an aliphatic saturated or unsaturated chain.
21. The PICK1 inhibitor according to any one of the preceding items, wherein the lipophilic aliphatic group is an aliphatic cycle comprising a gonane structure, such as sterol.
22. The PICK1 inhibitor according to any one of the preceding items, wherein the lipophilic aliphatic group is an aliphatic cycle comprising a steroid, such as cholesterol.
23. The PICK1 inhibitor according to any one of the preceding items, wherein the lipophilic aliphatic group further comprises a functional group, such as a carboxylic acid, an alcohol or an amine.
24. The PICK1 inhibitor according to any one of the preceding items, wherein the lipophilic aliphatic group comprises an alcohol.
25. The PICK1 inhibitor according to any one of the preceding items, wherein the lipophilic aliphatic group comprises a carboxylic acid.
26. The PICK1 inhibitor according to any one of the preceding items, wherein the lipophilic aliphatic group is a fatty acid.
27. The PICK1 inhibitor according to any one of the preceding items, wherein the lipophilic aliphatic group is a C4-026 fatty acid.
28. The PICK1 inhibitor according to any one of the preceding items, wherein the lipophilic aliphatic group is a saturated fatty acid or an unsaturated fatty acid.
29. The PICK1 inhibitor according to any one of the preceding items, wherein the lipophilic aliphatic group is selected from the group consisting of acetic acid, butyric acid, caproic acid, caprylic acid, capric acid, lauric acid, myristic acid, palmitic acid, margaric acid, stearic acid, arachidic acid, behenic acid, lignoceric acid, cerotic acid, caproleic acid, lauroleic acid, myristoleic acid, palmitoleic acid, oleic acid, linoleic acid, linolenic acid, gadoleic acid, erucic acid.
30. The PICK1 inhibitor according to any one of the preceding items, wherein the lipophilic aliphatic group is selected from the group consisting of capric acid, lauric acid, myristic acid, palmitic acid, margaric acid, stearic acid.
31. The PICK1 inhibitor according to any one of the preceding items, wherein the lipophilic aliphatic group is myristic acid.
32. The PICK1 inhibitor according to any one of the preceding items, wherein the lipophilic aliphatic group is a diacid, such as for example tetradecanedioic acid, hexadecanedioic acid, or octadecanedioic acid.
33. The PICK1 inhibitor according to any one of the preceding items, wherein the lipophilic aliphatic group is conjugated via a functional group, such as a carboxylic acid, to the nitrogen atom of the NPEG linker, such as by forming an amide.
34. The PICK1 inhibitor according to any one of the preceding items, wherein the non-peptide portion further comprises one amino acid.
35. The PICK1 inhibitor according to any one of items 14 to 15, wherein the one amino acid is an α-amino acid or a β-amino acid.
36. The PICK1 inhibitor according to any one of items 14 to 16, wherein the one amino acid is selected from the group consisting of Asp, β-Asp, β-Ser, β-homo-Ser and β-Lys.
37. The PICK1 inhibitor according to item 14, wherein the one amino acid is conjugated to the nitrogen atom of the NPEG linker via the α-carboxylic acid to form an amide and is further conjugated to the lipophilic aliphatic group.
38. The PICK1 inhibitor according to any one of items 14 to 17, wherein the one amino acid is conjugated to the lipophilic aliphatic group via the α- or β-amine to form an amide bond or via a side chain functional group, such as a carboxylic acid, an alcohol or an amine to form an amide bond or an ester bond.
39. The PICK1 inhibitor according to any one of the preceding items, wherein said PICK1 inhibitor has the generic structure of formula (I):

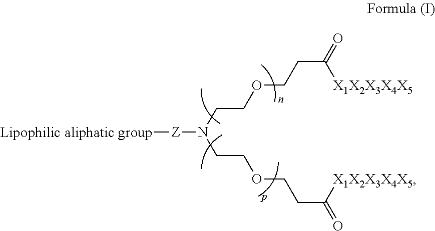

wherein
   Z is a bond or a single amino acid;
   n is an integer 0 to 12;
   p is an integer 0 to 12.
40. The PICK1 inhibitor according to any one of the preceding items, wherein the PICK1 inhibitor has a structure according to formula (II):

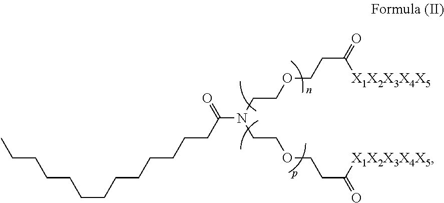

wherein
   n is an integer 0 to 12, preferably 2;
   p is an integer 0 to 12, preferably 2.
41. The PICK1 inhibitor according to any one of the preceding items, wherein the PICK1 inhibitor self-assembles into a higher order structure in solution, such as self-assemble to form micellar structures.
42. The PICK1 inhibitor according to any one of items 20 to 21, wherein higher order structure has a radius of gyration (R_{g}) of at least 15 Å, such as at least 17 Å, for example at least 19 Å, such as at least 20 Å, for example at least 21 Å, such as at least 22 Å, for example at least 23 Å, such as at least 24 Å, for example at least 25 Å, such as at least 26 Å, for example at least 27 Å, such as at least 28 Å, for example at least 29 Å, such as at least 30 Å, for example at least 31 Å.
43. The PICK1 inhibitor according to any one of the preceding items, wherein the PICK1 inhibitor binds to the PDZ domain of PICK1.
44. The PICK1 inhibitor according to any one of the preceding items, wherein the PICK1 inhibitor binds to the PDZ domain of two or more PICK1 proteins.
45. The PICK1 inhibitor according to any one of the preceding items, wherein the PICK1 inhibitor is capable of inhibiting a protein-protein interaction between PICK1 and AM PAR.
46. The PICK1 inhibitor according to any one of the preceding items, wherein the PICK1 inhibitor is capable of inhibiting PICK1.
47. The PICK1 inhibitor according to any one of the preceding items, wherein said peptide has a Ki for PICK1 inferior to 10 nM, such as inferior to 9 nM, such as inferior to 8 nM, such as inferior to 7 nM, such as inferior to 6 nM, such as inferior to 5 nM, such as inferior to 4 nM, such as inferior to 3 nM, such as inferior to 2 nM, such as inferior to 1 nM, such as inferior to 0.5 nM.
48. The PICK1 inhibitor according to any one of the preceding items, wherein binding of the PICK1 inhibitor to PICK1 result in formation of higher oligomeric states of PICK1, such as trimers, tetramers, pentamers, hexamers, heptamers or octamers of PICK1.
49. The PICK1 inhibitor according to any one of the preceding items, wherein binding of the PICK1 inhibitor to PICK1 result in formation of tetramers, hexamers or octamers of PICK1.
50. The PICK1 inhibitor according to any one of the preceding items, further comprising a detectable moiety.
51. The PICK1 inhibitor according to any one of the preceding items, wherein the detectable moiety is conjugated to the first and/or the second peptide.
52. The PICK1 inhibitor according to any one of the preceding items, wherein the detectable moiety is conjugated to the one amino acid of the non-peptide portion.
53. The PICK1 inhibitor according to item 27, wherein the detectable moiety is a fluorophore, a chromophore or an enzyme.
54. The PICK1 inhibitor according to item 27, wherein the detectable moiety is 5, 6-carboxyltetramethylrhodamine (TAMRA) or indodicarbocyanine (Cy5).
55. The PICK1 inhibitor according to item 27, wherein the detectable moiety comprises or consists of a radioisotope.
56. The PICK1 inhibitor according to item 30, wherein the radioisotope is selected from the group consisting of ¹²⁵I, ^{99m}Tc, ¹¹¹In, ⁶⁷Ga, ⁶⁸Ga, ⁷²As, ⁸⁹Zr, ¹²³I, ¹⁸F and ²⁰¹Tl.
57. A micelle comprising a PICK1 inhibitor according to any one of the preceding items.
58. A pharmaceutical composition comprising a PICK1 inhibitor according to any one of items 1 to 56 or the micelle according to item 57.
59. The PICK1 inhibitor, the micelle or the pharmaceutical composition according to any one of the preceding items for use as a medicament.
60. The PICK1 inhibitor, the micelle or the pharmaceutical composition according to any one of the preceding items, for use in the prophylaxis and/or treatment of a disease or disorder associated with maladaptive plasticity.
61. A method of providing prophylaxis and/or treatment of a disease or disorder associated with maladaptive plasticity in a subject, the method comprising administering the PICK1 inhibitor, the micelle or the pharmaceutical composition according to any one of the preceding items to the subject.
62. Use of the PICK1 inhibitor, the micelle or the pharmaceutical composition according to any of the preceding items for the manufacture of a medicament for the treatment of diseases and/or disorders associated with maladaptive plasticity.
63. The PICK1 inhibitor, the micelle or the pharmaceutical composition for use according to any one of items 59 to 60, the method according to item 61 or the use according to item 62, wherein the disease or disorder associated with maladaptive plasticity is pain, drug addiction, amyotrophic lateral sclerosis, epilepsy, tinnitus, migraine, cancer, ischemia, Alzheimer's disease, and/or Parkinson's disease.
64. The PICK1 inhibitor, the micelle or the pharmaceutical composition for use according to any one of items 59 to 60, the method according to item 61 or the use according to item 62, wherein the disease or disorder associated with maladaptive plasticity is pain, such as inflammatory pain or neuropathic pain.
65. The PICK1 inhibitor, the micelle or the pharmaceutical composition for use according to item 64, wherein the pain is mechanical or thermal allodynia or hyperalgesia.
66. PICK1 inhibitor, the micelle or the pharmaceutical composition for use according to any one of items 59 to 60, the method according to item 61 or the use according to item 62, wherein the disease or disorder associated with maladaptive plasticity is drug addiction, such as cocaine addiction, opioid addiction, or morphine addiction.
67. The PICK1 inhibitor, the micelle or the pharmaceutical composition for use according to any one of items 59 to 60, the method according to item 61 or the use according to item 62, wherein the disease or disorder associated with maladaptive plasticity is cancer such as breast cancer, for example histological grade, lymph node metastasis, Her-2/neu-positivity, and triple-negative basal-like breast cancer.
68. The PICK1 inhibitor, the micelle or the pharmaceutical composition for use according to any one of items 59 to 60, the method according to item 61 or the use according to item 62, wherein the disease or disorder associated with maladaptive plasticity is amyotrophic lateral sclerosis.
69. The PICK1 inhibitor, the micelle or the pharmaceutical composition for use according to any one of items 59 to 60, the method according to item 61 or the use according to item 62, wherein the disease or disorder associated with maladaptive plasticity is epilepsy.
70. The PICK1 inhibitor, the micelle or the pharmaceutical composition for use according to any one of items 59 to 60, the method according to item 61 or the use according to item 62, wherein the disease or disorder associated with maladaptive plasticity is tinnitus.
71. The PICK1 inhibitor, the micelle or the pharmaceutical composition for use according to any one of items 59 to 60, the method according to item 61 or the use according to item 62, wherein the disease or disorder associated with maladaptive plasticity is migraine.
72. The PICK1 inhibitor, the micelle or the pharmaceutical composition for use according to any one of items 59 to 60, the method according to item 61 or the use according to item 62, wherein the disease or disorder associated with maladaptive plasticity is ischemia.
73. The PICK1 inhibitor, the micelle or the pharmaceutical composition for use according to any one of items 59 to 60, the method according to item 61 or the use according to item 62, wherein the disease or disorder associated with maladaptive plasticity is Alzheimer's disease.
74. The PICK1 inhibitor, the micelle or the pharmaceutical composition for use according to any one of items 59 to 60, the method according to item 61 or the use according to item 62, wherein the disease or disorder associated with maladaptive plasticity is Parkinson's disease.
75. A PICK1 inhibitor according to any one of items 1 to 56 or a micelle according to item 57, for use in diagnosis of a disease or disorder associated with maladaptive plasticity.
76. The PICK1 inhibitor for use in diagnosis according to item 75, wherein the disease or disorder associated with maladaptive plasticity is cancer, such as breast cancer.
77. The PICK1 inhibitor for use according to item 76, wherein the breast cancer is selected from histological grade, lymph node metastasis, Her-2/neu-positivity, and triple-negative basal-like breast cancer.
78. A method of diagnosing breast cancer in a subject in need thereof, the method comprising the steps of:
   a. obtain a tissue sample from said subject;
   b. staining the sample with the PICK1 inhibitor according to items 50-56;
   c. determining the level of PICK1 in the sample; and
   d. comparing the level of PICK1 in the sample to a healthy standard,
   wherein an increased level of PICK1 in the sample is indicative of said individual having breast cancer.
79. A method for predicting the prognosis for a subject suffering from breast cancer, the method comprising the steps of:
   a. obtain a tissue sample from said subject;
   b. staining the sample with the PICK1 inhibitor according to items 50-56;
   c. determining the level of PICK1 in the sample; and
   d. comparing the level of PICK1 in the sample to a healthy standard,
   wherein an increased level of PICK1 in the sample is indicative of poor prognosis.

### EXAMPLES

### Example 1: PICK1 Expression and Purification

Full length rat PICK1 (pET41) was prepared as described earlier (Madsen et al., 2005). In brief, PICK1 was expressed in BL21-DE3-pLysS cells and grown at 37° C., induced at OD₆₀₀=0.6 with 1 mM Isopropyl β-D-1-thiogalactopyranoside (IPTG) and grown 16 hrs at 20° C. Cultures were harvested and re-suspended in 50 mM trisaminomethane (Tris), 125 mM NaCl, 2 mM Dithiothreitol (DTT, Sigma), 1% Triton X-100 (Sigma), 20 μg/mL DNAse 1 and ½ a tablet cOmplete protease inhibitor cocktail (Roche) pr. 1 L culture. The re-suspended pellets were frozen at −80° C. for later purification. The lysate was cleared by centrifugation (36,000×g for 30 min at 4° C.), and the supernatant was incubated with Glutathione-Sepharose 4B beads (GE Healthcare) for 2 hrs at 4° C. under gentle rotation and then centrifuged at 4,000×g for 5 min. The supernatant was removed and the beads were washed twice in 35 mL 50 mM Tris, 125 mM NaCl, 2 mM DTT and 0.01% Triton-X100. The beads were transferred to PD-10 Bio-Spine Chromatography columns (Bio-Rad) and washed with an additional 3 column volumes. Each column was sealed and 0.075 U/μL, Novagen® was added for cleavage 0/N at 4° C. under gentle rotation. PICK1 was eluted on ice and absorption at 280 nm was measured on TECAN plate reader or on a NanoDrop3000. The protein concentration was determined using lambert beers law (A=εcl), εA280PICK1=32320 (cm*mol/L)⁻¹.

### Example 2: Synthesis of Compounds

PEG₀-(HWLKV)₂, PEG₁-(HWLKV)₂, PEG₂-(HWLKV)₂, PEG₃-(HWLKV)₂, PEG₄-(HWLKV)₂, Ac-(HWLK_{PEG4}V)₂, and NPEG₄-(HWLKV)₂ were synthesized by solid phase peptide synthesis as described in Bach et al., 2012. NPEG₄-(HWLKV)₂ was myristoylated as described in Nissen et al., 2015 to provide myr-NPEG₄-(HWLKV)₂.

Fluorescently labelled peptides (5-FAM) were prepared by conjugation of 5-FAM directly to the amine of the NPEG-linker or by conjugation of 5-FAM to the N-terminus of the peptide via a 6-aminohexanoic acid (Ahx) linker.

### Example 3: Characterization of In-Solution Behavior of Myr-NPEG-(HWLKV)₂

To test the in-solution behavior of myr-NPEG₄-(HWLKV)₂ we did size exclusion chromatography (SEC) with comparison to the unconjugated NPEG₄-(HWLKV)₂ as a control. We also did Small Angle X-ray scattering (SAXS) of myr-NPEG₄-(HWLKV)₂ in solution to obtain an overall estimate of particle sizes and shape.

Materials and Methods:

Size exclusion chromatography: Size exclusion chromatography was done using an Äkta purifier with a Superdex200 Increase 10/300 column, where 500 μL of NPEG₄-(HWLKV)₂ or myr-NPEG₄-(HWLKV)₂ at the indicated concentration was injected onto the column. Absorbance profile was measured at 280 nm and plotted against elution volume using Graph Pad Prism 8.3.

Small Angle X-Ray Scattering:

Concentration series of myr-NPEG-(HWLKV)₂ ranging from 0.18 mg/ml to 9.37 mg/mL was prepared in buffer containing 50 mM Tris (pH 7.4), 125 mM NaCl. Samples were measured at the P12 SAXS Beamline, Petra III, DESY, Hamburg, Germany. Preliminary data reduction including radial averaging and conversion of the data into absolute scaled scattering intensity, I(q), as a function of the scattering vector q, where q=4π sin(θ)/λ (λ=half scattering angle) were done using the standard procedures at the beamline.

The modelling of the SAXS data was performed in two ways, firstly using the pair distance distribution function and subsequently using a molecular constrained core-shell model for polydisperse spheres. In brief, it was assumed that the peptide aggregated into polydisperse spherical micelles. Here, the hydrophobic tails form the core and these are surrounded by the hydrophilic part (the shell) in a spherical micelle. For the modelling, scattering lengths of 2.26e-10 cm and 2.96e-11 cm were used for the headgroups and tail, respectively. This was calculated by counting the number of electrons in the molecular structure and multiplying by the scattering length of the electron. For the scattering length density calculations, it was assumed that the molecular volume of the C₁₃ alkyl chain was 377 Å3 as estimated from Tanfords empirical formula (V=27.4+26.9n_{C}) where n_{C} denote the number of carbons. i.e. 13 in this case. The hydrophilic headgroup was as a first estimate assumed to have a mass density corresponding to that of a soluble protein (i.e. 1.35 g/cm³), this yielded a molecular volume of 2078 Å3. This value was taken as a free parameter in the fits and refined to a molecular volume of ˜2010 Å3 corresponding to a mass density of 1.39 g/cm³ and with very little variation over the fits to the different sample concentrations. Excess scattering length densities were then calculated in relation to water (9.4e101/cm²). Using this molecular restrained model, the volumes of the core and of the shell were coupled internally through the fitted aggregation number, N_{agg}. The polydispersity was described by a Gauss function of N_{agg}. The Gauss was truncated at ±3sigma. The model module “PolydisperseMicelles” from the WillItFit software was used for the fitting. As usual a small surface roughness and a small constant background was also necessary for the model to converge.

Scattering data was merged; buffer subtracted and binned using WillItRebin with a binning factor of 1.02. The pair distance distribution functions (pddf) were fitted using bayesapp (http://www.bayesapp.org/) in the interval [0.0122-0.4] Å⁻¹, fitting parameters are reported in the data collection table below.

| | |
|---|---|
| Sample details | Myr-NPEG₄-(HWLKV)₂ |
| Peptide | C₁₄-PEG₄-(HWLKV)₂ |
| Buffer | 50 mM Tris, 125 mM NaCl, pH 7.4 |
| Extinction coefficient | 11000 M⁻¹ cm⁻¹ |

| | | |
|---|---|---|
| Molecular weight | 1874 | g/mol |
| Peptide concentration | 0.19-9.37 | mg/ml |

SAXS data collection details

| | |
|---|---|
| Instrument | P12, Petra III, DESY [1] |
| Date for data collection | Sep. 5, 2019 |
| Wavelength (Å) | 1.24 |
| Measured q-range (Å⁻¹) | 0.0025-0.73 |
| Absolute calibration | Water |
| Exposure time (ms) | 10 |
| Temperature (K) | 293.35 |

Software

| Data rebin | WillItRebin | | | | | |
|---|---|---|---|---|---|---|
| Pair distance distribution | | | | | | |
| function derived results | | | | | | |
| Molecular weight (kDa) | 14.8 | 13.2 | 13.8 | 11.2 | 10.4 | 9.2 |
| n_{myr-NPEG4-(HWLKV)2} | 7.9 | 7.1 | 7.3 | 6.0 | 5.5 | 4.9 |
| Core shell model obtained | | | | | | |
| results | | | | | | |
| Concentration | 300 μM | 400 μM | 1000 μM | 3000 μM | 5000 μM | |
| n_{myr-NPEG4-(HWLKV)2} | 19 | 22 | 22 | 21 | 19 | |

Footnotes and references
[1] C. E. Blanchet, A. Spilotros, F. Schwemmer, M. A. Graewert, A. Kikhney, C. M. Jeffries, D. Franke, D. Mark, R. Zengerle, F. Cipriani, S. Fiedler, M. Roessle, D. I. Svergun, J Appl Crystallogr 2015, 48, 431.
[2] S. Hansen, Journal of Applied Crystallography 2012, 45, 566.
[3] S. Hansen, Journal of Applied Crystallography 2014, 47, 1469.

Results:

In this series of experiments, we have tested myr-NPEG₄-(HWLKV)₂ for its ability to assemble into higher order oligomeric structures using SEC and SAXS.

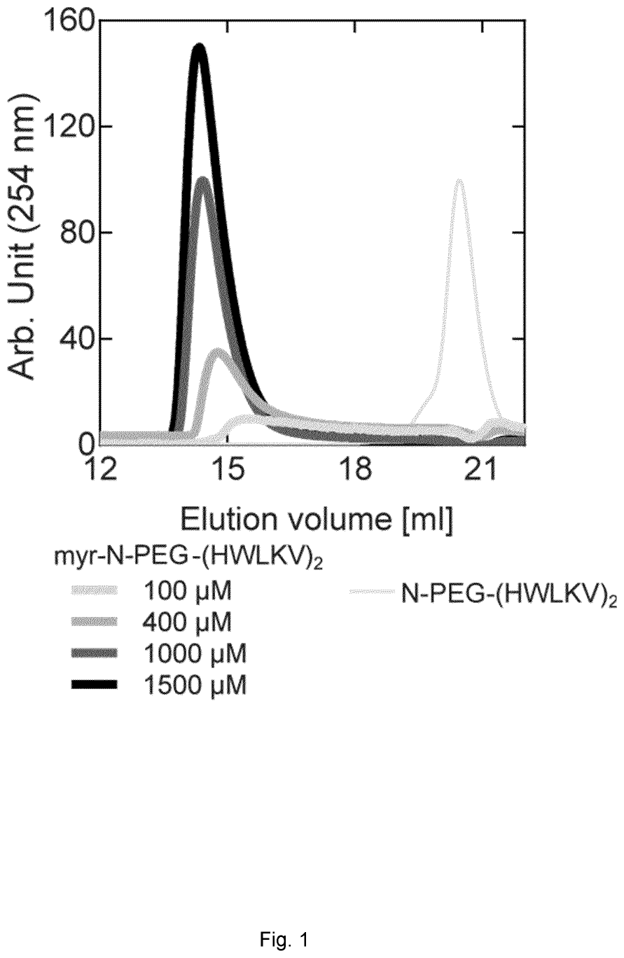

Size exclusion chromatography: SEC experiments were performed to show the concentration dependency on self-assembly of myr-NPEG₄-(HWLKV)₂ in comparison to the unconjugated NPEG-(HWLKV)₂ and found that myr-NPEG₄-(HWLKV)₂ eluted at lower elution volume, suggesting a larger hydrodynamic radius, and hence a larger molecular assembly (FIG. 1).

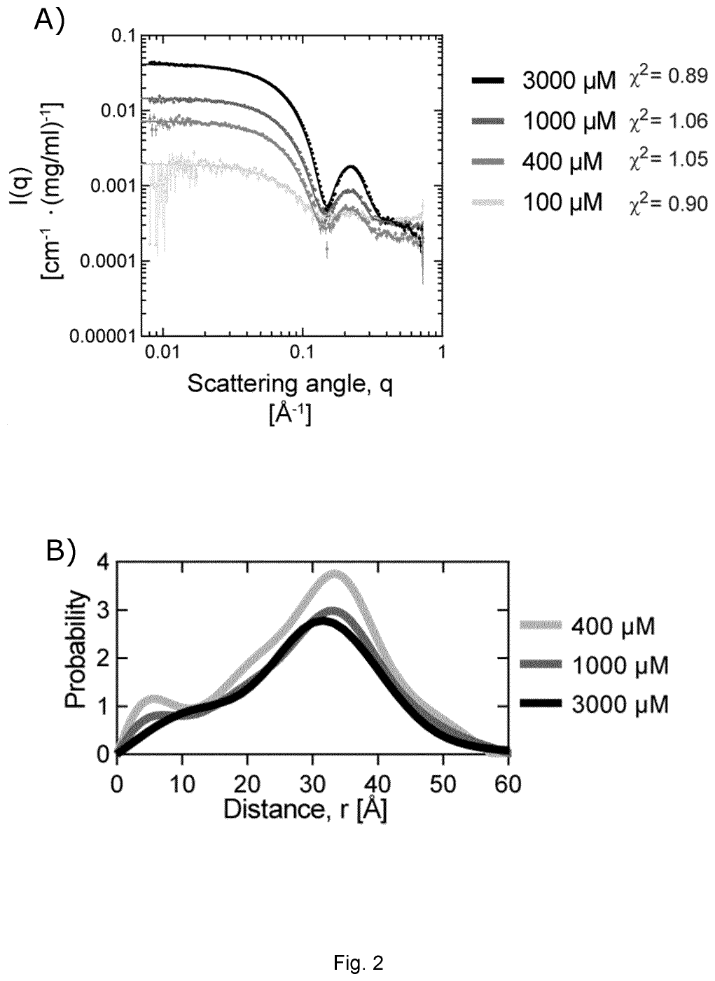

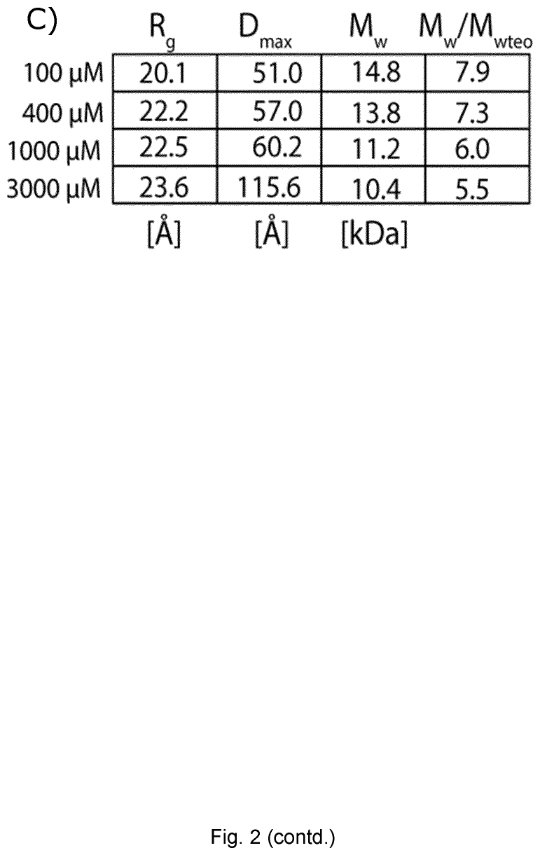

Small Angle X-ray scattering: SAXS experiments were conducted to show the concentration dependency on self-assembly of myr-NPEG₄-(HWLKV)₂ and it was found that using a standard analysis, analyzing the pair distance distribution function, myr-NPEG₄-(HWLKV)₂ apparently assembled into micellar structures with a radius of gyration (R_{g}) of 20-23.6 Å consisting of 5-8 individual molecules (FIG. 2a-c). Since the pair distance distribution function does not account for the negative scattering contribution of the aliphatic group, this analysis underestimates the number of molecules in the molecular assembly. Analysis of the data using a core-shell model as described above, suggested that myr-NPEG₄-(HWLKV)₂ assembled into a micellar structure, like a spherical core-shell shape with an outer radius of 20-24 Å comprising 19-22 individual myr-NPEG₄-(HWLKV)₂ molecules.

Conclusion

The present example demonstrates that the PICK1 inhibitor of the present disclosure is capable of forming higher order structures, such as micellar structures in solution.

### Example 4: Binding of Myr-NPEG₄-(HWLKV)₂ to PICK1

To test the binding of myr-NPEG₄-(HWLKV)₂ to recombinant PICK1 fluorescence polarization was performed. To validate the formation of higher order complexes of PICK1 upon binding, size exclusion chromatography was performed.

Materials and Methods

PICK1 was expressed and purified as described in Example 1.

Fluorescence polarization: Fluorescence polarization was carried out in competition mode at a fixed concentration of protein and tracer (5FAM-NPEG₄-(HWLKV)₂, 5 nM or 5FAM-HWLKV, 20 nM), against an increasing concentration of indicated unlabelled peptide. The plate was incubated for 2 hours on ice in a black half-area Corning Black non-binding surface 96-well plate. The fluorescence polarization was measured directly on an Omega POLARstar plate reader using excitation filter at 488 nm and long pass emission filter at 535 nm. The data was plotted using GraphPad Prism 8.3, and fitted to the One site competition, to extract K_{i,app} value.

Size exclusion chromatography: Size exclusion chromatography was performed using an Äkta purifier with a Superdex200 Increase 10/300 column, where 500 μL of 40 μM PICK1 in absence or presence of 10 μM myr-NPEG₄-(HWLKV)₂ was loaded to the column. Absorbance profile was measured at 280 nm and plotted against elution volume using Graph Pad Prism 8.3.

Results

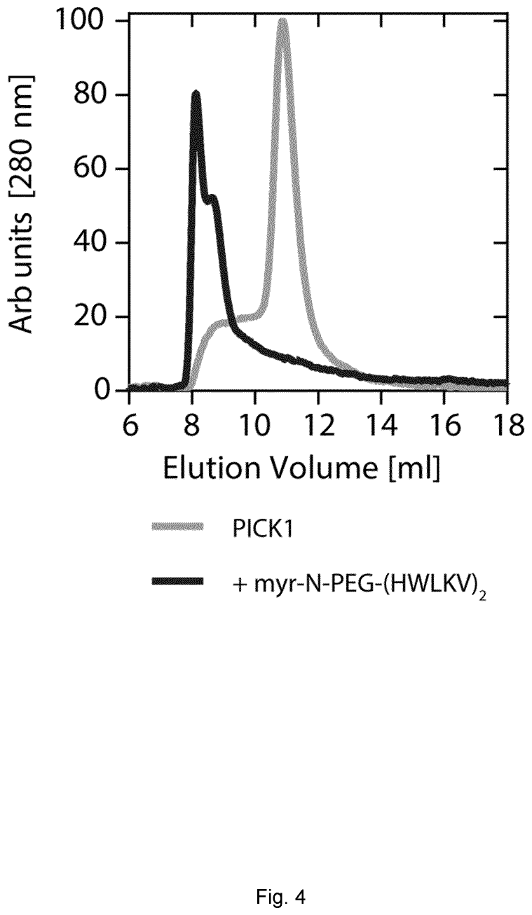

In this series of experiments, we have tested myr-NPEG₄-(HWLKV)₂ for its ability to bind to recombinant purified PICK1 using fluorescence polarization, and the ability of myr-NPEG₄-(HWLKV)₂ to induce higher order oligomers of PICK1 when in complex as evidenced by SEC (FIG. 4).

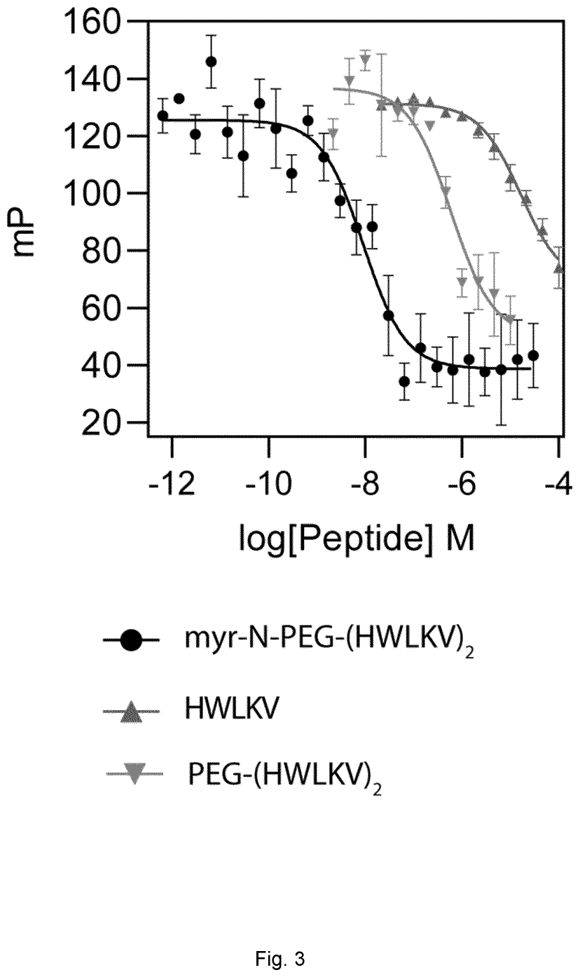

Fluorescence polarization (FP) experiments were performed to determine binding affinity for PICK1. Competition experiment, using 5FAM-NPEG₄-(HWLKV)₂ or 5FAM-HWLKV as fluorescent tracer, demonstrated a >1000-fold affinity increase of myr-NPEG₄-(HWLKV)₂ (Ki,app=3.0 nM, SEM interval [2.3-3.8] nM, n=6) compared to HWLKV (Ki,app=6998 nM, SEM interval [4972-9849] nM, n=3) and a >50-fold affinity increase of myr-NPEG₄-(HWLKV)₂ compared to NPEG₄-(HWLKV)₂ (Ki,app=179 nM, SEM interval [169-189], n=6) (FIG. 3).

Size exclusion chromatography was done in order to evaluate the in-solution behavior of myr-NPEG₄-(HWLKV)₂ in complex with PICK1. Elution volumes suggested that myr-NPEG₄-(HWLKV)₂, forms higher order oligomeric structures of PICK1 upon binding (FIG. 4).

Conclusion

The present example demonstrates that the PICK1 inhibitor of the present disclosure shows high affinity binding to PICK1. Conjugation of a lipid to the bivalent peptide ligand provides a >50-fold affinity increase as compared to the unconjugated NPEG₄-(HWLKV)₂.

The present example further demonstrates that the PICK1 inhibitor of the present disclosure is capable of inducing higher order structures of PICK1 upon binding. Inhibition of the protein function is likely to result from such induction of higher order structures of PICK1.

### Example 5: Optimizing the Sequence of the PICK1 Binding Peptide Ligand to Identify High Affinity Binders

To test the stringency of the PICK1 PDZ binding motif in the DAT-05 (HWLKV) sequence (i.e. position X₁-X₅) and to indicate putatively peptides with better affinity, we performed an initial study using fluorescence polarization binding to purified PICK1 of 95 different penta-peptides with each residue in the HWLKV sequence substituted to either of the 19 other natural amino acids.

Further, we took the data obtained in the above experiment, and utilized it for guidance to design 52 different penta-, tetra and tri-peptides, derived from combinatorial substitution of amino acids. To verify putative peptides with better affinity, binding affinities to purified PICK1 were studied by fluorescence polarization binding assays.

Materials and Methods:

Fluorescence polarization: Fluorescence polarization was carried out in competition mode at a fixed concentration of protein and tracer (5FAM-HWLKV, 20 nM), against an increasing concentration of indicated unlabeled peptide. The plate was incubated 20 min on ice in a black half-area Corning Black non-binding surface 96-well plate and the fluorescence polarization was measured directly on a Omega POLARstar plate reader using excitation filter at 488-nm and long pass emission filter at 535-nm. The data was plotted using GraphPad Prism 6.0, and fitted to the One site competition, to extract K_{d} values, which were all correlated to the HWLKV affinity, which was finally plotted.

Results

Single Substitution Experiment:

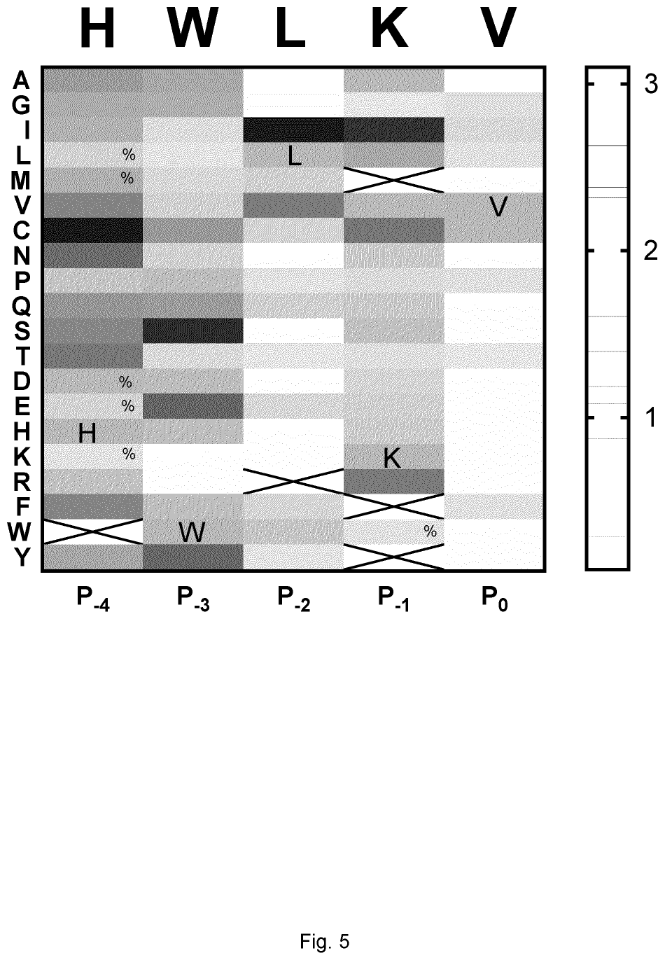

Substitution of X₁ and X₃ was mostly disruptive to binding (indicated by lighter shades) except for substitution of X₃ to V and I, which increased affinity (FIG. 5). On position X₂, substitutions to R, C, I and L all increased affinity and most substitutions were tolerated. Likewise, substitutions of X₄ and X₅ were in general well tolerated with notable exceptions of positively charged residues in X₄, which decreased affinity. Substitution to Y, E, S, Q, C, A and G in position X₄, increased affinity. Most substitutions (including Y, F, T, S, Q, N, C, V, M, I, G, A) increased affinity in position X₅ albeit several substitutions compromised solubility.

52 Combinatorial Peptides:

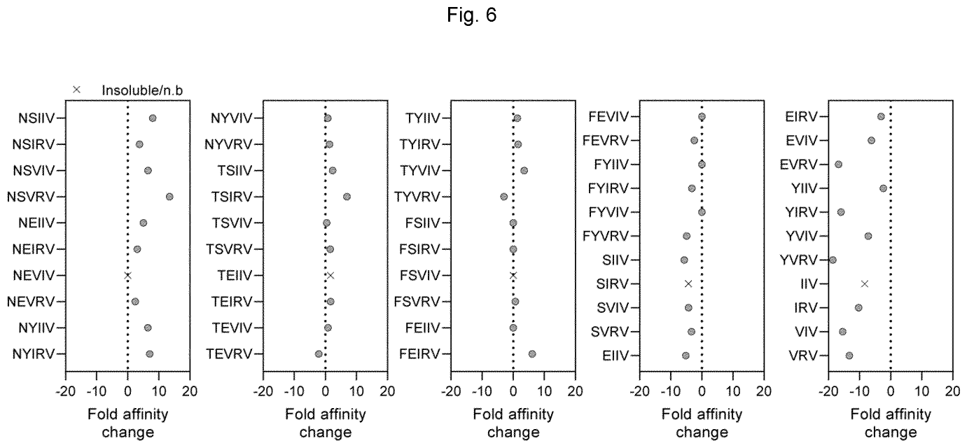

Based on double substitutions in class II binding motifs we found that many combinations were well tolerated, and in general N at position X₁, S/E at position X₂, R at position X₄ had a better or non-perturbed affinity, while F at position X₁ was, in general, not as well tolerated (FIG. 6).

Conclusion

This example demonstrates that optimization of the HWLKV sequence by amino acid substitutions provide peptide ligands showing equivalent and even higher affinity towards PICK1.

### Example 6: Variants of PEG and Variants of Attachment Site of PEG to Peptide Ligands

In this series of experiment, we wanted to test the affinity of various PEGₓ (x=0-4 ethylene glycol moieties) containing bivalent peptide ligands (FIG. 7) towards purified PICK1. In addition, we also wanted to test the affinity towards purified PICK1 when using a different linker attachment where PEG₄ is linked to the side chain of lysine (K) amino acid of sequence HWLKV (FIG. 7; ac-(HWLK_{PEG4}V)₂.

Materials and Methods

PICK1 was expressed and purified as described in Example 1.

Fluorescence polarization: Fluorescence polarization was carried out in competition mode at a fixed concentration of protein and tracer (5FAM-NPEG₄-(HWLKV)₂, 5 nM), against an increasing concentration of unlabelled PEGₓ-(HWLKV)₂. The plate was incubated 2-4 hrs on ice in a black half-area Corning Black non-binding surface 96-well plate and the fluorescence polarization was measured on a Omega POLARstar plate reader using excitation filter at 488-nm and long pass emission filter at 535-nm. The data was plotted using GraphPad Prism 6.0, and fitted to the One site competition, to extract Kₗ values, which were all correlated to the affinity of HWLKV peptide, which was finally plotted as fold affinity increase.

Results

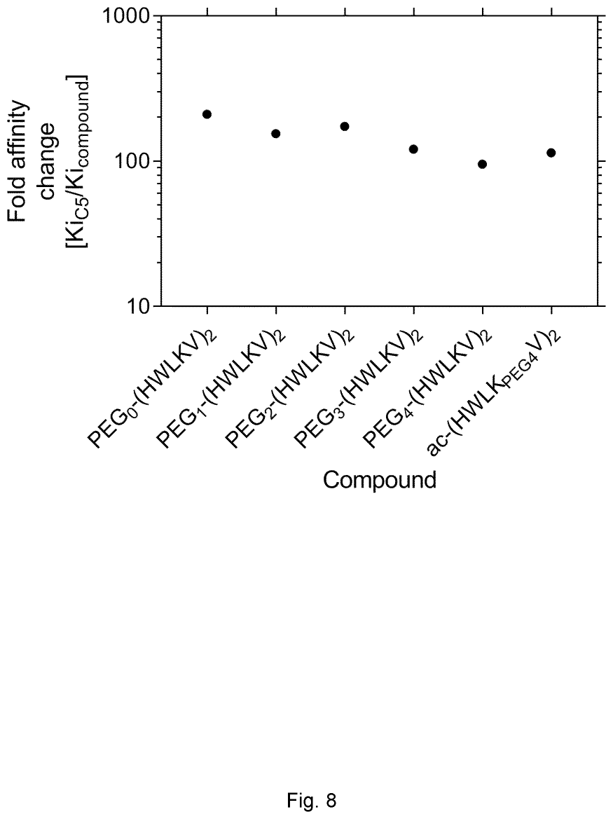

To test the minimal distance required between the two identical penta-peptides, we decided to test different PEG linker lengths and positions. We found little variation in affinity between different lengths of the PEG linker, all providing enhancing PICK1 affinity as compared to the HWLKV peptide (FIG. 8).

Furthermore, we found that attachment of the PEG₄ linker to the X₄ lysine side chain amine, instead of the N-terminal amine did not alter the affinity remarkably compared to PEG₄-HWLKV.

Conclusion

The present example demonstrates that variation of the length and attachment sites of the linker in the PICK1 inhibitor of the present disclosure is well tolerated.

### Example 7: Efficacy Assessment of Myr-NPEG₄-(HWLKV)₂ in Inflammatory Pain

In this series of experiments, we aim to assess the treatment efficacy of myr-NPEG₄-(HWLKV)₂ to relieve inflammatory pain in the Complete Freund's Adjuvant (CFA) model in mice.

Materials and Methods.

Inflammatory pain: Animals were habituated to the experimental room for a minimum of 60 min before initiation of the experiment. Mechanical pain threshold was determined by von Frey measurements of both hind paws. Injury was induced on the right hind paw, whereas the contralateral left hind paw was used as internal control of the animal. Von Frey filaments ranging from 0.04 to 2 g (g=gram-forces) (0.04, 0.07, 0.16, 0.4, 0.6, 1.0, 1.4, 2.0) were used for determination of mechanical pain threshold. In the current experiment filaments in ascending order were applied to the frontocentral plantar surface of the hind paws. Mice were placed in PVC plastic boxes (11.5 cm×14 cm) on a wire mesh, and allowed 30 min habituation prior of habituation. Each von Frey hair was applied five times with adequate resting periods between each application and number of withdrawals recorded. The withdrawal threshold was determined as the von Frey filament eliciting at least 3 positive trials out of the 5 applications in two consecutive filaments. A positive trial was defined as sudden paw withdrawal, flinching and/or paw licking induced by the filament. The inflammatory pain was induced by injection of 50 μL undiluted Complete Freund's Adjuvant (CFA) (F5881, Sigma) unilaterally into the intraplantar surface of the right hind paw, whereas control mice were injected with the same amount of 0.9% saline (B. Braun, Germany). All intraplantar injections were performed with and insulin needle (0.3 mL BD Micro-Fine) while the animal was under isoflurane anesthesia (2%) for maximum 60 seconds. Von Frey was applied up to 11 days after unilateral CFA injection depending on the experiment. The myr-NPEG₄-(HWLKV)₂ was administered through different routes (intrathecal (i.t. (7 μL)) or s.c. (10 μL/g)), and at different concentrations (2, 10 or 50 μmol/kg). Statistical analysis was performed using GraphPad Prism 6.0. Two-way RM ANOVA followed by Bonferroni's post-hoc test. Significance level set to p<0.05.

Results:

In the following are presented the efficacy of myr-NPEG₄-(HWLKV)₂ in relieving inflammatory pain in the CFA model using 3 different treatments.

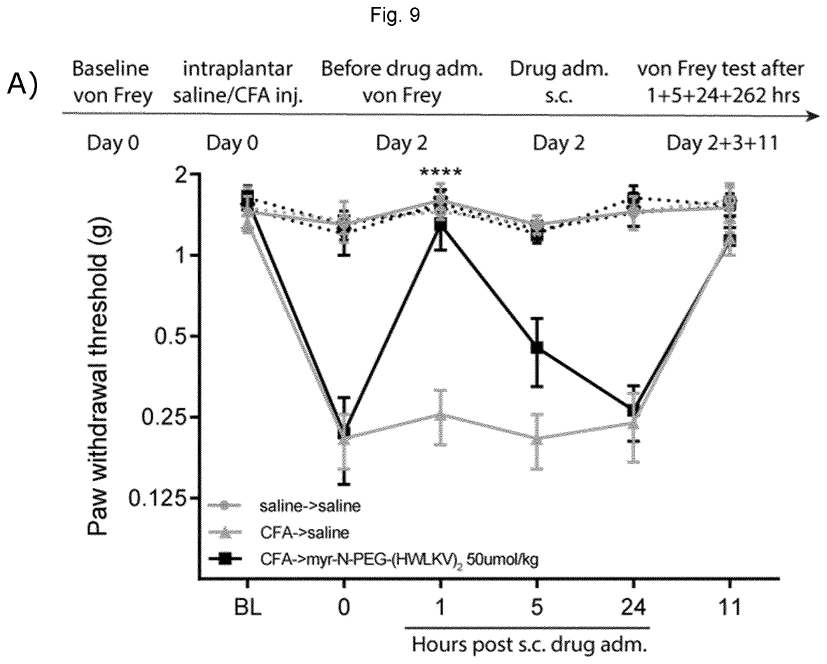

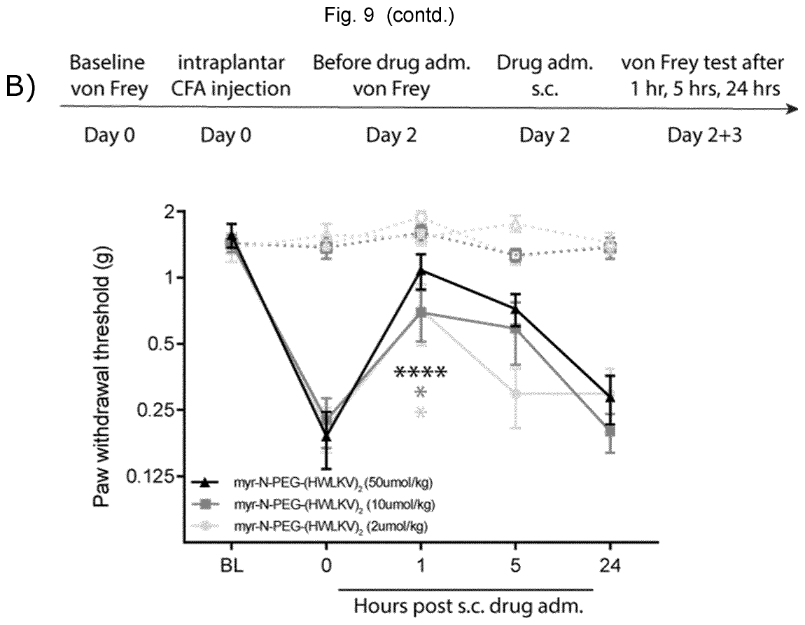

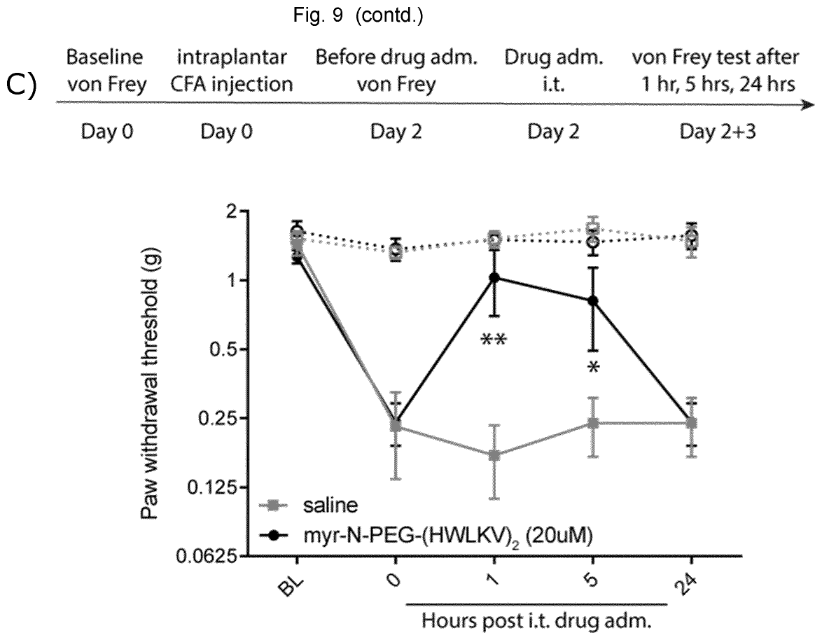

On day 0 mice were injected i.pl. into the right hind paw with 50 μL of CFA or saline. Two days later, mice were injected s.c. with 50 μmol/kg (10 μL/gram) myr-NPEG₄-(HWLKV)₂ or saline. Evoked pain was tested with the use of von Frey filaments before injection as well as 1, 5 and 24 hours after injection. Two-way ANOVA followed by Bonferroni's post-hoc analysis revealed an overall significant effect of myr-NPEG₄-(HWLKV)₂, with significant pain relief when tested 1 hour post injection. In addition, data reveals no effect of saline injection into the hind paw instead of CFA, and CFA effect on hyperalgesia is gone at 11 days post injection (FIG. 9a).

On day 0 mice were injected i.pl. into the right hind paw with 50 μL of CFA or saline. Two days later, hyperalgesia was confirmed by using von Frey filaments, and mice were injected s.c. with 2, 10 or 50 μmol/kg (10 μL/gram) myr-NPEG₄-(HWLKV)₂ or saline (10 μL/gram). Evoked pain was tested again with the use of von Frey filaments at 1, 5 and 24 hours after injection. Two-way ANOVA followed by Bonferroni's post-hoc analysis revealed an overall significant effect of myr-NPEG₄-(HWLKV)₂, with significant pain relief up to 5 hours post injection for the highest concentration tested, and significant pain relief when tested 1 hour post injection for the two lower concentrations (FIG. 9b).

On day 0 mice were injected i.pl. into the right hind paw with 50 μL of CFA or saline. Two days later, hyperalgesia was confirmed by using von Frey filaments, and mice were injected intrathecally with 20 μM myr-NPEG₄-(HWLKV)₂ or saline at a volume of 7 μL. Evoked pain was tested again with the use of von Frey filaments at 1, 5 and 24 hours after injection. Two-way ANOVA followed by Bonferroni's post-hoc analysis revealed an overall significant effect of myr-NPEG₄-(HWLKV)₂, with significant pain relief at 1 hour and 5 hours after myr-NPEG₄-(HWLKV)₂ administration and no effect 24 hours after administration (FIG. 9c).

Conclusion

This example demonstrates that in the CFA model of inflammatory pain, myr-NPEG₄-(HWLKV)₂ significantly alleviate inflammatory pain, as revealed by increased paw withdrawal threshold (FIGS. 9a-c). In addition, it was found that the paw withdrawal threshold remained unaltered in the uninjured contralateral paw. myr-NPEG₄-(HWLKV)₂ was efficient at alleviating pain following subcutaneous injection of two dosages (FIG. 9a-b). Furthermore, myr-NPEG₄-(HWLKV)₂ alleviated inflammatory pain after intrathecal injection (FIG. 9c), confirming that myr-NPEG₄-(HWLKV)₂ inhibits central sensitization.

### Example 8: Efficacy Assessment of Myr-NPEG₄-(HWLKV)₂ in Neuropathic Pain

In this experiment, we aim to assess the treatment efficacy of myr-NPEG₄-(HWLKV)₂ to relieve neuropathic pain in the Spared Nerve Injury (SNI) model in mice.

Materials and Methods

Neuropathic pain. The SNI pain experiment was performed by Phenotype Expertise—Pain and CNS behaviour CRO under supervision of Stéphane Gaillard, PhD (CEO). Study was performed on 8 weeks old C57Bl6J male mice (Charles River). The spared nerve injury surgery was performed on anaesthetized mice. Ligature and transection of the common peroneal and tibial distal branches of the sciatic nerve was performed, leaving the sural branch intact. 7 days post-surgery, a decrease of threshold response to von Frey filaments of ipsilateral hind-paw was confirmed by von Frey filaments, corresponding to neuropathic pain condition. The mechanical threshold response of the operated mice was measured with calibrated von Frey filaments (“up/down” method) and the 50% threshold (g) was calculated. The experimenter was blinded to mice treatment. Mechanical threshold was measured before surgery, and again on day 7 at 0 hrs, 1 hr, 2 hr, 3 hr, 4 hrs and 6 hrs post drug administration. All the compounds were diluted in PBS and administered s.c. at 10 μL/g. Statistical analysis was performed using GraphPad Prism 6.0. Two-way RM ANOVA followed by Bonferroni's post-hoc test. Significance level set to p<0.05.

Results:

Mice underwent surgery leading to partial nerve injury, by cutting of the peroneal and tibial nerves, producing hypersensitivity of the remaining sural nerve (SNI). Seven days later, hyperalgesia was confirmed by using von Frey filaments, and mice were injected s.c. with 2 or 10 μmol/kg (10 μL/gram) myr-NPEG₄-(HWLKV)₂ or saline (10 μL/gram mouse). Evoked pain was tested again with the use of von Frey filaments at 1, 2, 3, 4 and 6 hours after injection. Two-way ANOVA followed by Bonferroni's post-hoc analysis revealed an overall significant effect of myr-NPEG₄-(HWLKV)₂, with significant pain relief up to 3 hours post injection for the highest concentration tested, and no significant pain relief of the lower concentration.

Conclusion

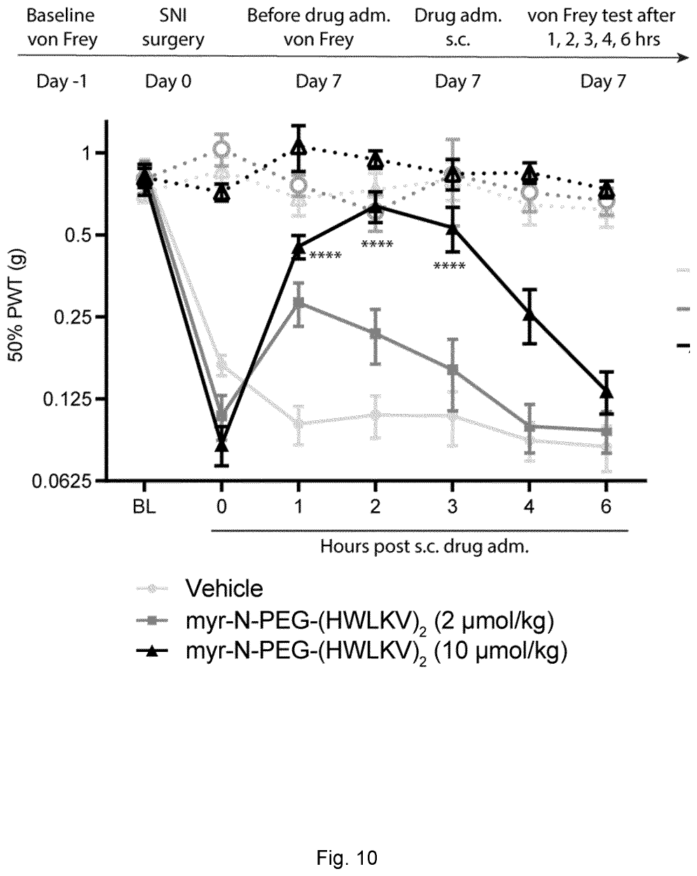

This example demonstrates that in the SNI model of neuropathic pain, myr-NPEG₄-(HWLKV)₂ significantly alleviate neuropathic pain, as revealed by increased paw withdrawal threshold in the mice following treatment. In addition, it was found that the paw withdrawal threshold remained unaltered in the uninjured contralateral paw. This confirms that myr-NPEG₄-(HWLKV)₂ is efficient at alleviating pain following subcutaneous injection in a dose-dependent manner (FIG. 10).

### Example 9: Efficacy Assessment of NPEG₄-(HWLKV)₂ in Neuropathic Pain

In this experiment, the aim was to assess the treatment efficacy of NPEG₄-(HWLKV)₂ (not possessing an aliphatic chain, PD5) to relieve neuropathic pain after the Spared Nerve Injury (SNI) model in mice.

Materials and Methods

Neuropathic pain. Study was performed on 8 weeks old C57Bl6J male mice (Charles River). The spared nerve injury surgery was performed on anaesthetized mice. Ligature and transection of the common peroneal and tibial distal branches of the sciatic nerve was performed, leaving the sural branch intact. 9 days post-surgery, a decrease of threshold response to von Frey filaments of ipsilateral hind-paw was confirmed by von Frey filaments, corresponding to neuropathic pain condition. The mechanical threshold response of the operated mice was measured with calibrated von Frey filaments (“up/down” method) and the 50% threshold (g) was calculated. The experimenter was blinded to mice treatment. Mechanical threshold was measured before surgery, and again on day 9 at 0 hrs, 1 hr, 2 hr, 3 hr, 4 hrs and 6 hrs post drug administration. All the compounds were diluted in PBS and administered 10 μmol/kg (10 μL/gram) NPEG₄-(HWLKV)₂s.c. at 10 μL/g. Statistical analysis was performed using GraphPad Prism 6.0. Two-way RM ANOVA followed by Bonferroni's post-hoc test. Significance level set to p<0.05.

Results:

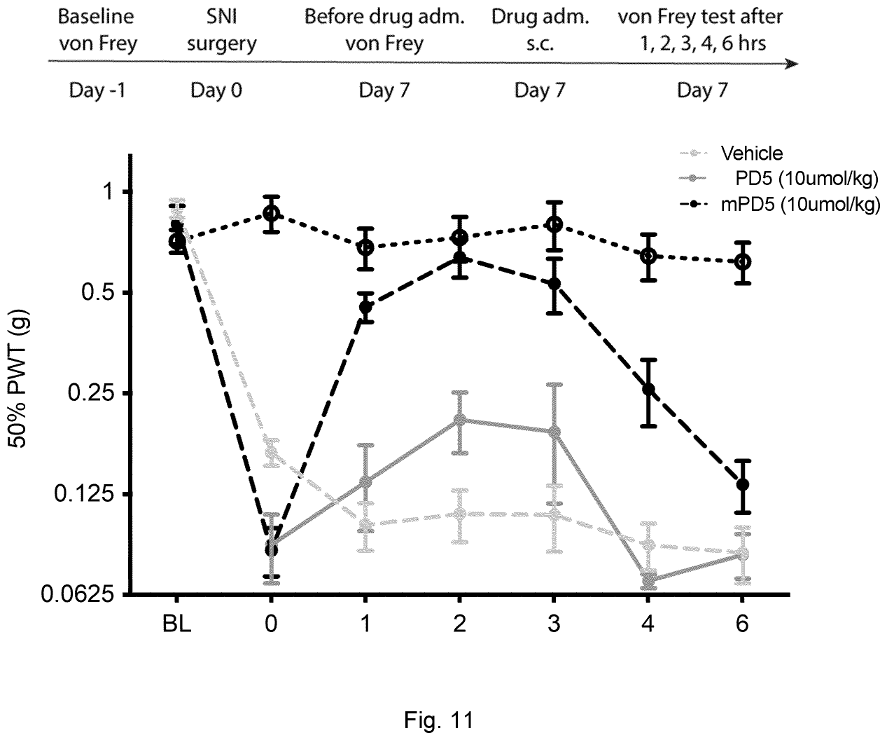

Mice underwent surgery leading to partial nerve injury, by cutting of the peroneal and tibial nerves, producing hypersensitivity of the remaining sural nerve (SNI). Nine days later, hyperalgesia was confirmed by using von Frey filaments, and mice were injected s.c. with 10 μmol/kg (10 μL/gram) NPEG₄-(HWLKV)₂ (PD5). Evoked pain was tested again with the use of von Frey filaments at 1, 2, 3, 4 and 6 hours after injection. Two-way ANOVA followed by Bonferroni's post-hoc analysis revealed no significant effect of treatment with NPEG₄-(HWLKV)₂ without the aliphatic group (FIG. 11). For comparison, myr-NPEG₄-(HWLKV)₂ and vehicle group from example 8 (FIG. 10) are shown in dashed lines.

Conclusion

This example demonstrates that in the SNI model of neuropathic pain, NPEG₄-(HWLKV)₂ (not possessing an aliphatic chain) does not significantly alleviate neuropathic pain, as revealed by increased paw withdrawal threshold in the mice following treatment.

### Example 10: Efficacy Assessment of Myr-NPEG₄-(HWLKV)₂ in Chronic Neuropathic Pain

In this experiment, the aim was to assess the treatment efficacy of myr-NPEG₄-(HWLKV)₂ to relieve neuropathic pain 1 year after the Spared Nerve Injury (SNI) model in mice.

Materials and Methods

Neuropathic pain. Study was performed on 8 weeks old C57Bl6J male mice (Charles River). The spared nerve injury surgery was performed on anaesthetized mice. Ligature and transection of the common peroneal and tibial distal branches of the sciatic nerve was performed, leaving the sural branch intact. 2 days post-surgery, a decrease of threshold response to von Frey filaments of ipsilateral hind-paw was confirmed by von Frey filaments, corresponding to neuropathic pain condition. The mice were testes in von Frey monthly and the hyperalgesic response at 52 weeks was sustained.

The mechanical threshold response of the operated mice was measured with calibrated von Frey filaments (“up/down” method) and the 50% threshold (g) was calculated. The experimenter was blinded to mice treatment. After injection, mechanical threshold was measured, 2 and 5 hrs. All the compounds were diluted in PBS and administered s.c. at 10 μL/g, 30 μmol/kg. Statistical analysis was performed using GraphPad Prism 6.0. One-way ANOVA followed by Dunnett's multiple comparisons test. ****, p<0.0001.

Results:

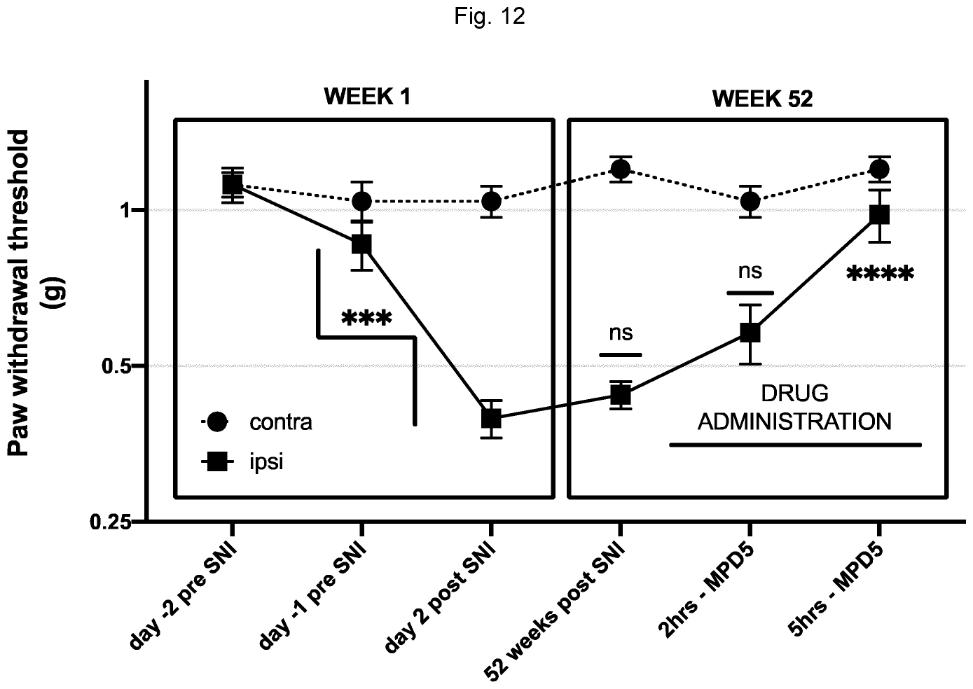

Mice underwent surgery leading to partial nerve injury, by cutting of the peroneal and tibial nerves, producing hypersensitivity of the remaining sural nerve (SNI). 2 days later, hyperalgesia was confirmed by using von Frey filaments (FIG. 12). This was unaltered after 52 weeks. Mice were injected s.c. with 30 μmol/kg (10 μL/gram) myr-NPEG₄-(HWLKV)₂ (10 μL/gram mouse). Evoked pain was tested again with the use of von Frey filaments at 2 and 5 hours after injection. One-way ANOVA followed by Dunnett's multiple comparisons test revealed a highly significant effect of the treatment at 5 hrs (p<0.0001)

Conclusion

This example demonstrates that in the SNI model of neuropathic pain, myr-NPEG₄-(HWLKV)₂ significantly alleviates neuropathic pain a full year after induction of the SNI injury, as revealed by increased paw withdrawal threshold in the mice following treatment.

### Example 11: Efficacy Assessment of Myr-NPEG₄-(HWLKV)₂ in Diabetic Neuropathy

In this experiment, the aim was to assess the treatment efficacy of myr-NPEG₄-(HWLKV)₂ to relieve diabetic neuropathy using the streptozocin (STZ) model of type1 diabetes.

Materials and Methods

Diabetic neuropathy (STZ) model. Diabetes is induced by a single IP injection of 200 μg/mL. Streptozocin solution (100 μl/10 g, Sigma-aldrich S0130, batch #WXBB7152V). Glycemia is tested before, and 7 days after injection. All injected mice present blood glucose concentration >350 mg/dL at D+7, and then are used for analgesic testing of the compounds at D+14. One mouse had to be euthanized at 7 days post-injection.

The mechanical threshold response of the operated mice was measured with calibrated von Frey filaments (“up/down” method) and the 50% threshold (g) was calculated. The experimenter was blinded to mice treatment. 13 days post-surgery, a decrease of threshold response to von Frey filaments of ipsilateral hind-paw was confirmed by von Frey filaments, corresponding to diabetic neuropathy After injection of myr-NPEG₄-(HWLKV)₂, mechanical threshold was measured, 1,2,4 and 6 hrs and again at day 15. Compounds were diluted in PBS (vehicle) and administered s.c. at 10 μL/g, in doses as indicated (gabapentine, 5MPK). Statistical analysis was performed using GraphPad Prism 6.0. One-way ANOVA followed by Dunnett's multiple comparisons test. ****, p<0.0001.

Results:

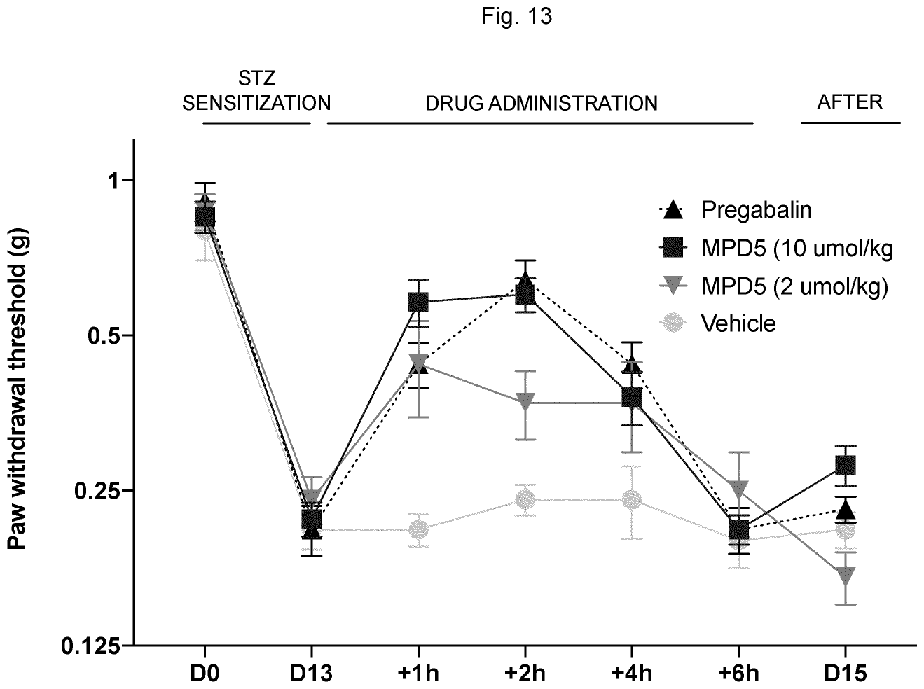

Seven days after STZ administration, all mice presented a drastic increase of glycemia (from 197.4+/−4.4 mg/dL to 533.5+/−10.4; see annex) validating the diabetic state of mice. As shown in FIG. 13, 13 days after STZ injection, diabetes-induced neuropathic pain is clearly established with a decrease of mechanical response threshold (mechanical allodynia). Pregabalin (5MPK) administration induced a significant increase of the mechanical response threshold compared to vehicle group (p<0.001 at 1 h, 2 h and 4 h post administration) with a maximum reversal up to 84.7±10.5% of baseline at +2 h. Both myr-NPEG₄-(HWLKV)₂ (mPD5) solutions at 2 and 10 μmol/kg induced a significant increase of the mechanical response threshold compared to vehicle group (p=0.006 at 1 h and p=0.003 at 2 h for the lowest concentration; p<0.001 at 1 h and 2 h, p<0.006 at 4 h for the 10 μmol/kg solution). The maximum reversal was reached after 2 h post-administration (49.2±7.9% and 67.3±4.5% of baseline, respectively for the 2 and 10 μmol/kg solutions). Interestingly, we also observed a statistical difference between the two groups treated with mPD5 at 1 h and 2 h (p=0.013 and p=0.008 respectively).

Conclusion

In this study, we have tested the analgesic effect of the myr-NPEG₄-(HWLKV)₂ on STZ-induced neuropathic pain model in mice. Interestingly, we have observed a strong and long-lasting effect up to 4 h after administration at 10 μmol/kg. In the same time, we also observed a dose-dependent effect of the peptide, with a weaker effect with 2 μmol/kg dose. As a note, no visible side effect was observed.

### Example 12: Efficacy Assessment of Myr-NPEG₄-(NSVRV)₂, Myr-NPEG₄-(SVRV)₂ and Myr-NPEG₄-(LRV)₂ in Inflammatory Pain

In this series of experiments, the aim was to assess the treatment efficacy of variants to the PDZ domain binding sequence as defines by the peptide optimization described in example 5 to relieve inflammatory pain in the Complete Freund's Adjuvant (CFA) model in mice. This defines translation from the screening in example 5 to in vivo efficacy and demonstrate effect of shorter binding motifs (C4 and C3).

Materials and Methods.

Inflammatory pain (CFA model): Animals were habituated to the experimental room for a minimum of 60 min before initiation of the experiment. Mechanical pain threshold was determined by von Frey measurements of both hind paws. Injury was induced on the right hind paw, whereas the contralateral left hind paw was used as internal control of the animal.

Von Frey filaments ranging from 0.04 to 2 g (g=gram-forces) (0.04, 0.07, 0.16, 0.4, 0.6, 1.0, 1.4, 2.0) were used for determination of mechanical pain threshold. In the current experiment filaments in ascending order were applied to the frontocentral plantar surface of the hind paws. Mice were placed in PVC plastic boxes (11.5 cm×14 cm) on a wire mesh, and allowed 30 min habituation prior of testing. Each von Frey hair was applied five times with adequate resting periods between each application and number of withdrawals recorded. The withdrawal threshold was determined as the von Frey filament eliciting at least 3 positive trials out of the 5 applications in two consecutive filaments. A positive trial was defined as sudden paw withdrawal, flinching and/or paw licking induced by the filament.

The inflammatory pain was induced by injection of 50 μL undiluted Complete Freund's Adjuvant (CFA) (F5881, Sigma) unilaterally into the intraplantar surface of the right hind paw, whereas control mice were injected with the same amount of 0.9% saline (B. Braun, Germany). All intraplantar injections were performed with an insulin needle (0.3 mL BD Micro-Fine) while the animal was under isoflurane anesthesia (2%) for maximum 60 seconds. Von Frey was applied up to 6 days after unilateral CFA injection depending on the experiment. The peptides were administered s.c. (10 μL/g)), and at different concentrations (0.4 and 2 μmol/kg). Statistical analysis was performed using GraphPad Prism 6.0. Two-way RM ANOVA followed by Dunnett's post-hoc test. Significance level set to p<0.05.

Results:

On day 0 mice were injected i.pl. into the right hind paw with 50 μL of CFA or saline. On day 2 after CFA injection, mice were injected s.c. with 0.4 μmol/kg (10 μL/gram) myr-NPEG₄-(HWLKV)₂, myr-NPEG₄-(NSVRV)₂, myr-NPEG₄-(SVRV)₂ or myr-NPEG₄-(LRV)₂ and on day 5 they were injected s.c. with 2 μmol/kg (10 μL/gram) myr-NPEG₄-(HWLKV)₂, myr-NPEG₄-(NSVRV)₂, myr-NPEG₄-(SVRV)₂ or myr-NPEG₄-(LRV)₂. At this concentration myr-NPEG₄-(NSVRV)₂ was not fully dissolved. On both days 2 and 5, evoked pain was tested with the use of von Frey filaments before injection as well as 1, 5 and 24 hours after injection. Two-way ANOVA for each day of administration separately followed by Dunnett's post-hoc analysis revealed the highest affinity compound, myr-NPEG₄-(NSVRV)₂, relieved pain at an S.c. dose of 0.4 μmol/kg, and further both myr-NPEG₄-(HWLKV)₂ and myr-NPEG₄-(SVRV)₂ relieved pain at S.c. dose of 2.0 μmol/kg (Figure XX). At this concentration myr-NPEG₄-(NSVRV)₂ was not fully dissolved. myr-NPEG₄-(LRV)₂ showed a tendency for pain relief at this dose but this was not significant in the experiment (FIG. 14).

Conclusion

This example demonstrates that in the CFA model of inflammatory pain, myr-NPEG₄-(NSVRV)₂, myr-NPEG₄-(SVRV)₂ or myr-NPEG₄-(LRV)₂ similar to myr-NPEG₄-(HWLKV)₂ alleviate inflammatory pain at 2 μmol/kg (10 μL/gram) as revealed by increased paw withdrawal threshold (FIG. 14). This confirms efficacy of shorter PICK1 PDZ-binding motifs (C4 and C3) in addition to the C5 PICK1 PDZ binding motif and moreover is in concordance with the affinities observed in example 5. Further to this, myr-NPEG₄-(NSVRV)₂ carrying the highest affinity peptide (NSVRV) of the optimization (example 5), demonstrated a significant pain relief at 0.4 μmol/kg (10 μL/gram).

### Example 13: Efficacy Assessment of Myr-NPEG₄-(HWLKV)₂ in Relief of Spontaneous Inflammatory Pain

The aim of this experiment was to assess the ability of myr-NPEG₄-(HWLKV)₂ to relieve not just evoked pain from the experimenter touching the inflamed paw but also to relieve the ongoing pain, spontaneous pain using single exposure place preference. This is considered paramount for clinical translation.

Materials and Methods.

Inflammatory pain (CFA model): Animals were habituated to the experimental room for a minimum of 60 min before initiation of the experiment. Mechanical pain threshold of both hind paws was determined by von Frey measurements. Injury was induced on the right hind paw, whereas the contralateral left hind paw was used as internal control of the animal. Von Frey filaments ranging from 0.04 to 2 g (g=gram-forces) (0.04, 0.07, 0.16, 0.4, 0.6, 1.0, 1.4, 2.0) were used for determination of mechanical pain threshold. In the current experiment filaments in ascending order were applied to the frontocentral plantar surface of the hind paws. Mice were placed in PVC plastic boxes (11.5 cm×14 cm) on a wire mesh, and allowed 30 min habituation prior of testing. Each von Frey filament was applied five times with adequate resting periods between each application and number of withdrawals recorded. The withdrawal threshold was determined as the von Frey filament eliciting at least 3 positive trials out of the 5 applications in two consecutive filaments. A positive trial was defined as sudden paw withdrawal, flinching and/or paw licking induced by the filament. The inflammatory pain was induced by injection of 50 μL undiluted Complete Freund's Adjuvant (CFA) (F5881, Sigma) unilaterally into the intraplantar surface of the right hind paw. All intraplantar injections were performed with an insulin needle (0.3 mL BD Micro-Fine), while the animal was under isoflurane anesthesia (2%) for maximum 60 seconds. Von Frey was prior of CFA injection, 2 days after CFA injection, and 5 days after CFA injection, confirming hypersensitivity both prior and after the Single exposure place preference experiment described below. Statistical analysis: was performed using GraphPad Prism 6.0. Student's t-test. Significance level set to p<0.05.

Single exposure place preference (sePP): sePP experiments were performed in a three-compartment rectangular apparatus consisting of a neutral zone (11.5×24 cm) in the middle, and two elongated compartments (28×24 cm) at the ends with different floor textures as well as different wall patterns. During the exposure sessions, the compartments were separated from each other by two off-white Plexiglas® partitions (24×40 cm), and on the test day, those partitions were removed.

The sePP protocol was conducted over three days, with exposure sessions on the two first days, and a test on the third day. On all three days, mice were allowed to habituate to the room for at least 60 min before initiation of the experiment. mPD5 was always paired with the compartment with grey walls and a punched floor, which has previously been shown to be the least preferred compartment. All mice from a cage were tested at the same time, but not all given the same treatment.

On exposure days, mice were weighed and injected s.c. with peptide (30 μmol/kg) or vehicle (10 μL/g) and immediately placed into the designated compartment for 60 min. The test group was exposed to peptide in the least preferred compartment, and vehicle (PBS) in the preferred compartment, whereas the control group was injected with vehicle in both compartments.

For the preference test on day 3, the Plexiglas® was removed, and mice could freely move between the three compartments for 20 minutes. Time spent in the different compartments was measured by Ethovision (Noldus, Wageningen, Netherlands). An additional experiment was run on animals without injury, showing no mPD5-dependent preference change

Results:

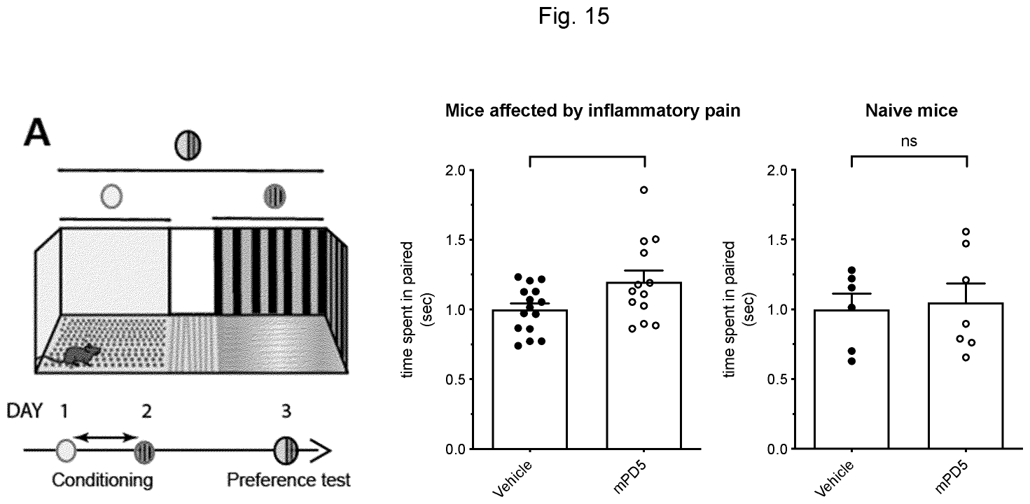

A single exposure to myr-NPEG₄-(HWLKV)₂ is sufficient to change the place preference of CFA-injured animals. At the preference test, myr-NPEG₄-(HWLKV)₂-treated animals spent significantly more time in the myr-NPEG₄-(HWLKV)₂-paired compartment, as compared to the vehicle-treated control mice (FIGS. 15A and B) indicating a preference for that compartment of the animals treated with myr-NPEG₄-(HWLKV)₂. To make sure, the change is preference is due to pain-relief and not that the peptide itself changes their preference, the exact same experiment was run on mice without injury (Naïve), showing no change in preference on the myr-NPEG₄-(HWLKV)₂-treated animals. This further indicates lack of abuse liability of myr-NPEG₄-(HWLKV)₂.

Conclusion:

This example demonstrates that mice with inflammatory pain shift their preference towards the chamber in which they have previously received myr-NPEG₄-(HWLKV)₂ demonstrating that mice perceive the drug to relief the ongoing pain, spontaneous pain. This is considered paramount for translational potential since most of the patient distress relate to ongoing pain.

### Example 14: Plasma Concentration of mPD5 at Different Concentrations Compared to Tat-PD5

The aim of this experiment was to assess plasma concentration and lifetime of myr-NPEG₄-(HWLKV)₂ and determine whether the acylation of myr-NPEG₄-(HWLKV)₂ extend plasma half-life in comparison to the parent molecule Tat-NPEG₄-(HWLKV)₂.

Materials and Methods:

Exposure of myr-NPEG₄-(HWLKV)₂ The mPD5 curves were determined by WUXI, DMPK and done by S.c. injection of 3 Male C57BL/6N Mouse (Fasted) with each concentration of mPD5 (2, 10 and 50 μmuol/kg) in sterile PBS and blood-samples were taken at times 30 min, 1 h, 2 h, 5 h, 12 h and plasma was subjected to LC-MS. Three point on the down-slope was determined from 3 points on the elimination phase.

Determination of Plasma Exposure of Biotinylated Tat-NPEG₄-(HWLKV)₂.

Blood sample collection. Biotinylated TAT-di-PEG4-DATC5 (biot-TPD5; 34 mg/kg=10 μmol) or Biotinylated myr-di-PEG4-DATC5 (biot-mPD5; 10 μmol) diluted in 0.9% NaCl was injected s.c in 8 weeks old male C57bl6N mice (18 mice in total) once, and blood samples were collected after 15 min, 30 min, 1 h, 2 h and 6 h (blood samples from 6 mice per timepoint) using Aprotinine-containing BD Vacutainer®K3EDTA tubes (BD Diagnostics). The blood samples were centrifuged at 3500 RPM for 15 min at 4° C. and the plasma was collected in new tubes and freezed down at −20° C.

ELISA. A 96 well NUNC IMMOBILIZER (Cat. No. 436006) plate was coated with 15 μg/mL biotinylated albumin (Sigma-Aldrich, Product no. A8549) diluted in 100 mM sodium carbonate buffer (pH 9.6) and incubated for 2 h at room temperature on shaker. After incubation the plate was washed with PBS-T (1× phosphate buffered saline with 0.1% TWEEN® 20 (Sigma-Aldrich)) 3×, 1 washing step overnight at room temperature on shaker. Pre-diluted HRP-conjugated Streptavidin (DAKO, Ref. no. P0397, 0.83 g/L) with 0.1% PBST (1:5000) was mixed with biot-TPD5 at different dilutions (10× and 20×) in a separate 96 well plate with round bottom (Thermo Scientific) and incubated 20 minutes on a shaker. The solution (100 μL/well) was loaded to the coated 96 well plate and incubated for 1 h at room temperature. After incubation the plate was washed with PBS-T 3× and developed in 100 μL TMB plus (Sigma, SLBT4708, T0440—1 L) in 3-5 min. The development was stopped by addition of 100 μL 0.2M sulphuric acid (H2SO4). The plate was read at 450 nm (and 570 nm) on a Wallac VICTOR2 1420 Multilabel Counter from PerkinElmer (Hvidovre, Denmark). The measured absorbance was calibrated to a standard curve generated from standard dilutions of biot-TPD5.

Results:

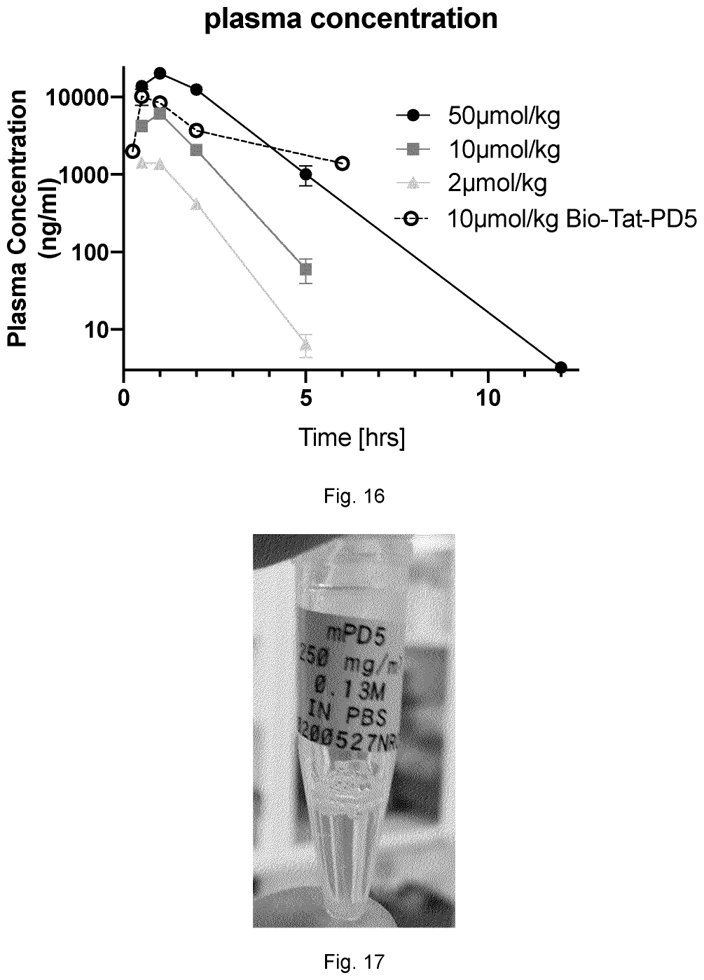

For myr-NPEG₄-(HWLKV)₂, we observed an initial increase in plasma concentration reaching maximal concentration after 1 h (1446±106 ng/ml after 3.75 mg/kg (2 μmol/kg) injection; 6173±508 ng/ml after 18.8 mg/kg (10 μmol/kg) injection; 20258±642 ng/ml after 93.8 mg/kg (50 μmol/kg) injection) followed by linear elimination phase on the semi-log scale indication 1 order kinetic. The maximal dose and area under the curve both scaled linearly with dose with T_{1/2} (0.50±0.07 h after 3.75 mg/kg (2 μmol/kg) injection; 0.59±0.07 h after 18.8 mg/kg (10 μmol/kg) injection; 0.84±0.03 h after 93.8 mg/kg (50 μmol/kg) injection) (FIG. 16). For biotinylated Tat-NPEG₄-(HWLKV)₂ (10 μmol/kg), we observed an increase in plasma concentration with a peak at 30 min followed by a non-linear elimination profile which slowed down at the late phase. Maximal concentration is similar to that observed for myr-NPEG₄-(HWLKV)₂ (10 μmol/kg) (FIG. 16).

Conclusion:

myr-NPEG₄-(HWLKV)₂ distributes to the plasma after S.c. administration in a dose dependent manner and is eliminated with 1. order kinetics and a half-life of 30-45 minutes. This is similar to Tat-NPEG₄-(HWLKV)₂ in agreement with behavioral effects demonstrating that the acylation on myr-NPEG₄-(HWLKV)₂ does not exert its effect by increasing plasma exposure or life-time.

### Example 15: Assessment of Solubility and Stability of Myr-NPEG₄-(HWLKV)₂

The objective was to determine the solubility, which is important for the preferred (subcutaneous) route of administration as well as chemical stability, which is critical for shelf-life of myr-NPEG₄-(HWLKV)₂

Materials and Methods:

Solubility was determined by visual inspection of samples dissolved in increasing concentration in 10 mM PBS. Stability was addressed by REDGLEAD for four concentration 2, 20, 50 and 200 μM, by leaving myr-NPEG₄-(HWLKV)₂ in PBS at 5 and degrees for 30 days followed by HPLC-UV-MS method

Results:

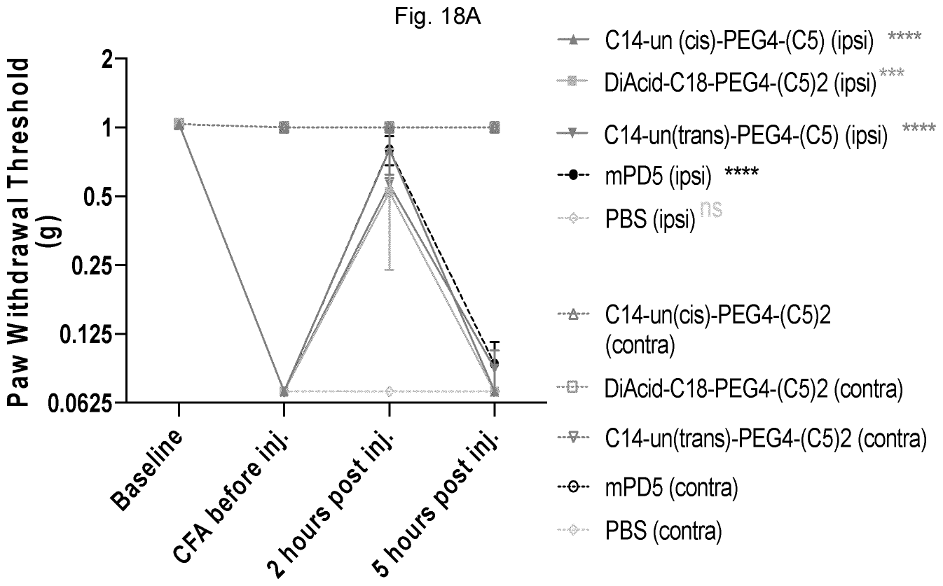

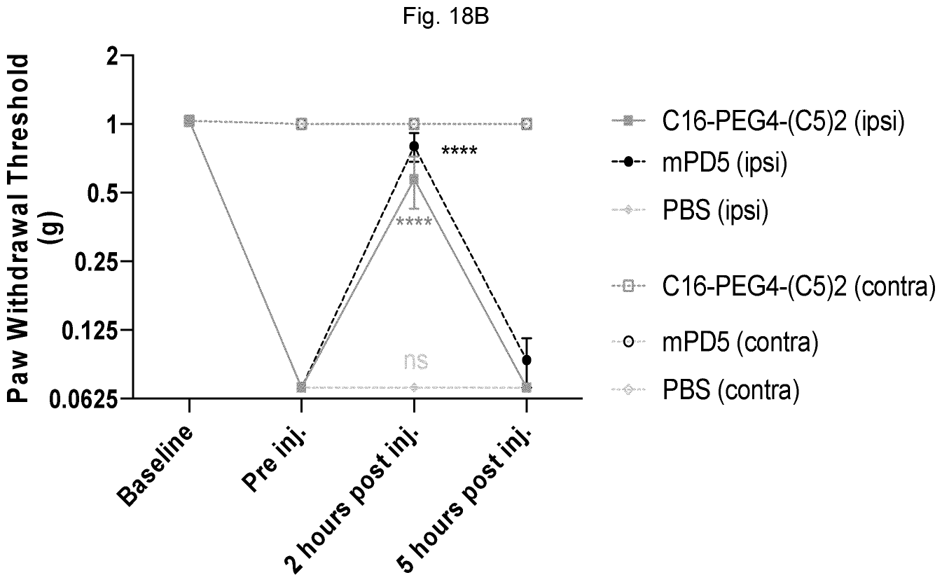

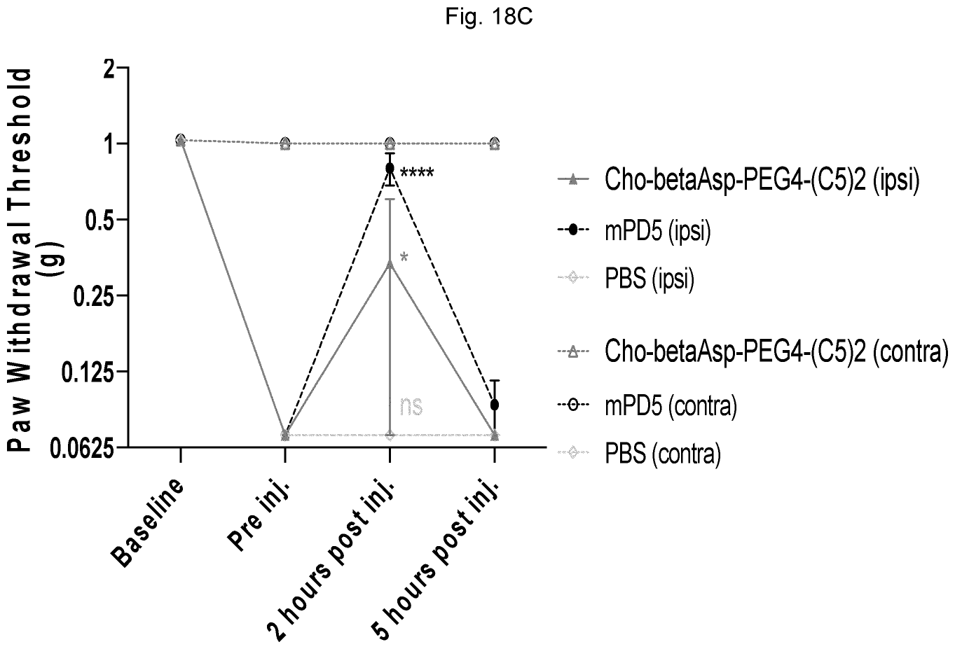

By visual inspection we determined the solubility of myr-NPEG₄-(HWLKV)₂ to be at least 250 mg/ml (130 mM) (FIG. 17); likely due to the micellar structure. No degradation was observed and the purity was determined to >98% for the samples with 200 and 50 μM. Due to the low absorption for the samples with 20 and 2 μM the purity could not be determined with certainty, even though no degradation could be found in either of these samples. The peptide mPD5 is chemically stable for at least 30 days in +5° C. and +25° C. in the vehicle compositions. The mass was confirmed for all samples including the standard samples for the calibration curve.

| | Nominel Conc. | | UV-HPLC | Measured Conc. |
|---|---|---|---|---|
| | mPD5 (μM) | pH | Peak area | mPD5 (μM) |
| 30 days | 200 | 7.37 | 6665 | 194 |
| @5° C | 50 | 7.39 | 3347 | 49 |
| | 20 | 7.40 | 1196 | 17 |
| | 2 | 7.37 | 37 | 0.5 |
| | 0 | | ND | <0.2 |
| 30 days | 200 | 7.36 | 7005 | 203 |
| @25° C | 50 | 7.41 | 3325 | 48 |
| | 20 | 7.39 | 1161 | 17 |
| | 2 | 7.37 | 26 | 0.4 |
| | 0 | | ND | <0.2 |

Conclusion:

myr-NPEG₄-(HWLKV)₂ demonstrates very good solubility, which is considered sufficient for human subcutaneous dosing (800 μl maximal volume) with the observed efficacy. Further to this, the stability is good, suggesting that the compound is compatible with shelf-life requirements.

### Example 16: Variation of Lipophilic Aliphatic Chain

In this series of experiments, the aim was to assess the treatment efficacy and prolongation of action by substation of the aliphatic chain in the Complete Freund's Adjuvant (CFA) model in mice.

Materials and Methods.

Inflammatory pain (CFA model): The inflammatory pain was induced by injection of 50 μL undiluted Complete Freund's Adjuvant (CFA) (F5881, Sigma) unilaterally into the intraplantar surface of the right hind paw, whereas control mice were injected with the same amount of 0.9% saline (B. Braun, Germany). All intraplantar injections were performed with an insulin needle (0.3 mL BD Micro-Fine) while the animal was under isoflurane anesthesia (2%) for maximum 60 seconds. Injury was induced on the right hind paw, whereas the contralateral left hind paw was used as internal control of the animal.

Animals were habituated to the experimental room for a minimum of 60 min before initiation of the experiment. Mechanical pain threshold was determined by von Frey measurements of both hind paws. Von Frey filaments ranging from 0.04 to 2 g (g=gram-forces) (0.04, 0.07, 0.16, 0.4, 0.6, 1.0, 1.4, 2.0) were used for determination of mechanical pain threshold. In the current experiment filaments in ascending order were applied to the frontocentral plantar surface of the hind paws. Mice were placed in PVC plastic boxes (11.5 cm×14 cm) on a wire mesh, and allowed 30 min habituation prior of testing. Each von Frey hair was applied five times with adequate resting periods between each application and number of withdrawals recorded. The withdrawal threshold was determined as the von Frey filament eliciting at least 3 positive trials out of the 5 applications in two consecutive filaments. A positive trial was defined as sudden paw withdrawal, flinching and/or paw licking induced by the filament. Von Frey was applied up to 5 days after unilateral CFA injection depending on the experiment. The peptides were administered s.c. (10 μL/g in PBS)), and at 2 μmol/kg with 5 injection of each compound in a cross-over schedule to assess their efficacy in relieving inflammatory pain. Statistical analysis was performed using GraphPad Prism 8.0. Two-way RM ANOVA followed by Dunnett's post-hoc test. Significance level set to p<0.05.

Results:

On day 0 mice baseline paw withdrawal response was determined using von frey filaments prior to intraplantar injection into the right hind paw with 50 μL of CFA or saline.

On day 2-5 after CFA injection mice were injected s.c. PBS or with 2.0 μmol/kg (10 μL/gram) of myr(C14)-NPEG₄-(HWLKV)₂, myr(C14) (un-saturated, trans)-NPEG₄-(HWLKV)₂, myr(C14) (un-saturated, trans)-NPEG₄-(HWLKV)₂, (C18) (diacid)-NPEG₄-(HWLKV)₂, (C16)-NPEG₄-(HWLKV)₂, Cholesterol-13-Asp-NPEG₄-(HWLKV)₂. On all days, evoked pain was tested with the use of von Frey filaments before injection as well as 2 and 5 hours after injection. Two-way ANOVA for each day of administration separately followed by Dunnett's post-hoc analysis.

Injection of PBS did not elicit any change in pain withdrawal threshold, whereas myr(C14)-NPEG₄-(HWLKV)₂ (mPD5) elicited a full and highly significant pain relief (FIG. 18A). Similarly, myr(C14) (un-saturated, trans)-NPEG₄-(HWLKV)₂, myr(C14) (un-saturated, cis)-NPEG₄-(HWLKV)₂, (C18) (diacid)-NPEG₄-(HWLKV)₂, elicited highly significant pain relief. Likewise, extension of the acyl chain to (C16)-NPEG₄-(HWLKV)₂ gave rise to highly significant pain relief (FIG. 18B). Finally, Cholesterol-β-Asp-NPEG₄-(HWLKV)₂ gave rise to a significant pain relief (FIG. 18C).

Conclusion

This example demonstrates that in the CFA model of inflammatory pain, unsaturation can be introduced to the acyl chain as can an additional acid group making it a diacid. Further to this, an acyl chain of 14 to 16 carbons and 18 (with a diacid) appear equivalent with respect to efficacy. Finally, the conjugation of the lipophilic aliphatic group (in this case cholesterol) via β-Asp display activity.

### Example 17: Efficacy of mPD5 on Relief of Sensitized Thermal Pain

In this experiment, the aim was to assess the treatment efficacy mPD5 of the sensitization of thermal pain sensation elicited by intraplantar injection of complete Freuds Adjuvans (CFA).

Materials and Methods.

Inflammatory pain (CFA model): Animals were habituated to the experimental room for a minimum of 60 min before initiation of the experiment. Hargreave's test was performed by application of radiant heat light to the plantar surface of both hindpaw. The response latency was measured by an automated readout (Ugo Basile, Italy). The baseline paw withdrawal latency of both hind paws in response to radiant heat stimulation was performed before CFA injection, and no difference between the two pre-selected groups was found. The inflammatory pain was induced by 10 injection of 50 μL undiluted Complete Freund's Adjuvant (CFA) (F5881, Sigma) unilaterally into the intraplantar surface of the right hind paw, whereas control mice were injected with the same amount of 0.9% saline (B. Braun, Germany). All intraplantar injections were performed with and insulin needle (0.3 mL BD Micro-Fine) while the animal was under isoflurane anesthesia (2%) for maximum 60 seconds. On day 3 after CFA injection, mice were placed in individual red cylinders (8 cm in diameter, 7.5 cm tall) and thermal hyperalgesia was confirmed by a baseline reading with IR of 20. Three measurements were performed on each hindpaw of each mouse. A positive trial was defined as sudden paw withdrawal, flinching and/or paw licking induced by the infrared light. Measurements were performed before CFA injection, and at 3+4 days after CFA injection. At day 3, the measurements were performed before treatment, as well as 1, 5 and 21 hours after treatment. Peptide and vehicle were administered s.c. (10 μL/g)), and at a concentration of 0 or 10 μmol/kg. Statistical analysis was performed using GraphPad Prism 6.0. Two-way RM ANOVA followed by Bonferroni's post-hoc test. Significance level set to p<0.05.

Results:

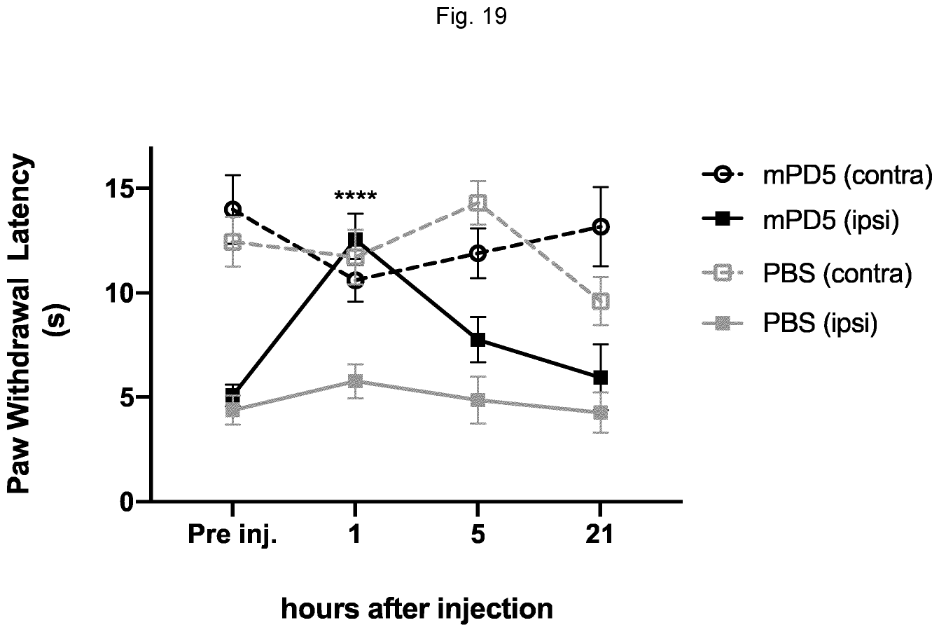

On day 3 after intraplantar CFA injection, thermal hyperalgesia was confirmed in the injected hindpaw (ipsilateral) using the Hargreave's test. Following s.c. administration of 10 μmol/kg (10 μL/gram) of myr-NPEG₄-(HWLKV)₂(mPD5), we observed a highly significant increase in paw withdrawal latency on the sensitized paw, whereas no significant changes was observed on the contralateral (healthy) paw. The pain relief was less pronounced at 5 hours and fully dissolved on the following day (21 h). Similar injection of PBS (vehicle) did not affect Paw withdrawal latency of either paw (FIG. 19).

Conclusion:

This example demonstrates that mPD5 can fully relief the thermal hypersensitivity observed in the CFA model of inflammatory pain, without affecting thermal sensitivity in the unaffected, healthy paw. This broadens the action to an important modality of pain innervated by a distinct subset of nociceptors from the transmitting hypersensitivity to touch.

Sequences

| SEQ ID NO: | Sequence |
|---|---|
| SEQ ID NO: 1 | X₁X₂X₃X₄X₅, wherein: |
| | X₁ is H, N, F, or T, or is absent; |
| | X₂ is W, S, E, or Y; or is absent; |
| | X₃ is L, V, or I; |
| | X₄ is K, I, or R; and |
| | X₅ is V; |
| SEQ ID NO: 2 | X₁X₂X₃X₄X₅, wherein: |
| | X₁ is N, F, or T, or is absent; |
| | X₂ is S, E, or Y; or is absent; |
| | X₃ is V, L or I; |
| | X₄ is I or R; and |
| | X₅ is V |
| SEQ ID NO: 3 | X₁X₂X₃X₄X₅, wherein: |
| | X₁ is N or T, or is absent; |
| | X₂ is S, E, or Y; or is absent; |
| | X₃ is V, L or I; |
| | X₄ is I or R; and |
| | X₅ is V |
| SEQ ID NO: 4 | X₁X₂X₃X₄X₅, wherein: |
| | X₁ is N or F, or is absent; |
| | X₂ is S, E, or Y; or is absent; |
| | X₃ is V, L or I; |
| | X₄ is I or R; and |
| | X₅ is V |
| SEQ ID NO: 5 | NSIIV |
| SEQ ID NO: 6 | NSIRV |
| SEQ ID NO: 7 | NSVIV |
| SEQ ID NO: 8 | NSVRV |
| SEQ ID NO: 9 | NEIIV |
| SEQ ID NO: 10 | NEIRV |
| SEQ ID NO: 11 | NEVIV |
| SEQ ID NO: 12 | NEVRV |
| SEQ ID NO: 13 | NYIIV |
| SEQ ID NO: 14 | NYIRV |
| SEQ ID NO: 15 | NYVIV |
| SEQ ID NO: 16 | NYVRV |
| SEQ ID NO: 17 | TSIIV |
| SEQ ID NO: 18 | TSIRV |
| SEQ ID NO: 19 | TSVIV |
| SEQ ID NO: 20 | TSVRV |
| SEQ ID NO: 21 | TEIIV |
| SEQ ID NO: 22 | TEIRV |
| SEQ ID NO: 23 | TEVIV |
| SEQ ID NO: 24 | TEVRV |
| SEQ ID NO: 25 | TYIIV |
| SEQ ID NO: 26 | TYIRV |
| SEQ ID NO: 27 | TYVIV |
| SEQ ID NO: 28 | TYVRV |
| SEQ ID NO: 29 | FSIIV |
| SEQ ID NO: 30 | FSIRV |
| SEQ ID NO: 31 | FSVIV |
| SEQ ID NO: 32 | FSVRV |
| SEQ ID NO: 33 | FEIIV |
| SEQ ID NO: 34 | FEIRV |
| SEQ ID NO: 35 | FEVIV |
| SEQ ID NO: 36 | FEVRV |
| SEQ ID NO: 37 | FYIIV |
| SEQ ID NO: 38 | FYIRV |
| SEQ ID NO: 39 | FYVIV |
| SEQ ID NO: 40 | FYVRV |
| SEQ ID NO: 41 | SIIV |
| SEQ ID NO: 42 | SIRV |
| SEQ ID NO: 43 | SVIV |
| SEQ ID NO: 44 | SVRV |
| SEQ ID NO: 45 | EIIV |
| SEQ ID NO: 46 | EIRV |
| SEQ ID NO: 47 | EVIV |
| SEQ ID NO: 48 | EVRV |
| SEQ ID NO: 49 | YIIV |
| SEQ ID NO: 50 | YIRV |
| SEQ ID NO: 51 | YVIV |
| SEQ ID NO: 52 | YVRV |
| SEQ ID NO: 53 | NSLRV |
| SEQ ID NO: 54 | HWLKV |
| N/A | IIV |
| N/A | IRV |
| N/A | VIV |
| N/A | VRV |
| N/A | LRV |

N/A: Not applicable

### REFERENCES

ALFONSO, S., et al., Synapto-depressive effects of amyloid beta require PICK1. Eur J Neurosci, 2014. 39(7): p. 1225-33.
ATIANJOH, F. E., et al., Spinal cord protein interacting with C kinase 1 is required for the maintenance of complete Freund's adjuvant-induced inflammatory pain but not for incision-induced post-operative pain. Pain, 2010. 151(1): p. 226-34.
BACH, A. et al. (2009). Design and synthesis of highly potent and plasma-stable dimeric inhibitors of the PSD-95-NMDA receptor interaction. Angew Chem Int Ed Engl 48, 9685-9689.
BACH, A et al., 2012. A high-affinity, dimeric inhibitor of PSD-95 bivalently interacts with PDZ1-2 and protects against ischemic brain damage. Proc Natl Acad Sci USA, 109, 3317-22.
BLANCHET, C. E. et al. J. Appl. Crystallogr. 2015, 48, 431.
CHEN, S. R., et al., Nerve injury increases GluA2-lacking AMPA receptor prevalence in spinal cords: functional significance and signaling mechanisms. J Pharmacol Exp Ther, 2013. 347(3): p. 765-72.
CLEM, R. L., V. Anggono, and R. L. Huganir, PICK1 regulates incorporation of calcium-permeable AMPA receptors during cortical synaptic strengthening. J Neurosci, 2010. 30(18): p. 6360-6.
CONRAD, K. L., et al., Formation of accumbens GluR2-lacking AMPA receptors mediates incubation of cocaine craving. Nature, 2008. 454(7200): p. 118-21.
DIXON, R. M., J. R. Mellor, and J. G. Hanley, Pick1-mediated glutamate receptor subunit 2 (GLUR2) trafficking contributes to cell death in oxygen/glucose deprived hippocampal neurons. Journal of Biological Chemistry, 2009.
GANGADHARAN, V., et al., Peripheral calcium-permeable AMPA receptors regulate chronic inflammatory pain in mice. J Clin Invest, 2011. 121(4): p. 1608-23.
GARRY, E. M., et al., Specific involvement in neuropathic pain of AMPA receptors and adapter proteins for the GluR2 subunit. Mol. Cell Neurosci., 2003. 24(1): p. 10-22.
HANSEN S. et al. Journal of Applied Crystallography 2012, 45, 566.
HANSEN S. et al. Journal of Applied Crystallography 2014, 47, 1469.
HE, J., et al., PICK1 inhibits the E3 ubiquitin ligase activity of Parkin and reduces its neuronal protective effect. Proc Natl Acad Sci USA, 2018. 115(30): p. E7193-E7201.
JI, R. R., et al., Central sensitization and LTP: do pain and memory share similar mechanisms? Trends Neurosci, 2003. 26(12): p. 696-705.
KANDASAMY, R. and T. J. Price, The pharmacology of nociceptor priming. Handb Exp Pharmacol, 2015. 227: p. 15-37.
KARLSEN, M. L. et al. 2015. Structure of Dimeric and Tetrameric Complexes of the BAR Domain Protein PICK1 Determined by Small-Angle X-Ray Scattering. Structure 23, 1258-70.
LONG, J. F. et al. Supramodular structure and synergistic target binding of the N-terminal tandem PDZ domains of PSD-95. J. Mol. Biol. 2003, 327, 203-214.
LORGEN, J O., PICK1 facilitates lasting reduction of GluA2 concentration in the hippocampus during chronic epilepsy. Epilepsy Res, 2017. 137: 25-32
LUSCHER, C. and R. C. Malenka, Drug-evoked synaptic plasticity in addiction: from molecular changes to circuit remodeling. Neuron, 2011. 69(4): p. 650-63.
MADSEN, K. L. et al., 2005. Molecular determinants for the complex binding specificity of the PDZ domain in PICK1. Journal of Biological Chemistry 280, 20539-20548.
MARCOTTE, D. J., et al., Lock and chop: A novel method for the generation of a PICK1 PDZ domain and piperidine-based inhibitor co-crystal structure. Protein Sci, 2018. 27(3): p. 672-680.
MOLLER, A. R., Tinnitus and pain. Prog Brain Res, 2007. 166: p. 47-53.
NISSEN et al. Design, synthesis, and characterization of fatty acid derivatives of a dimeric peptide-based postsynaptic density-95 (PSD-95) inhibitor. J. Med. Chem. 2015 Feb. 12; 58(3):1575-80.
PEKER, S. and A. Sirin, Parallels between phantom pain and tinnitus. Med Hypotheses, 2016. 91: p. 95-97.
TURNER et al. Administration of a novel high affinity PICK1 PDZ domain inhibitor attenuates cocaine seeking in rats. Neuropharmacology, 164(1), 2020, 107901.
VANNESTE, S., W. T. To, and D. De Ridder, Tinnitus and neuropathic pain share a common neural substrate in the form of specific brain connectivity and microstate profiles. Prog Neuropsychopharmacol Biol Psychiatry, 2019. 88: p. 388-400.
VIKMAN, K. S., B. K. Rycroft, and M. J. Christie, Switch to Ca2+-permeable AMPA and reduced NR2B NMDA receptor-mediated neurotransmission at dorsal horn nociceptive synapses during inflammatory pain in the rat. J Physiol, 2008. 586(2): p. 515-27.
WANG, W., et al., Preserved acute pain and impaired neuropathic pain in mice lacking protein interacting with C Kinase 1. Mol Pain, 2011. 7: p. 11.
WOLF, M. E. and C. R. Ferrario, AMPA receptor plasticity in the nucleus accumbens after repeated exposure to cocaine. Neurosci Biobehav Rev, 2010. 35(2): p. 185-211.
WOOLF, C. J. and M. W. Salter, Neuronal plasticity: increasing the gain in pain. Science, 2000. 288(5472): p. 1765-9.
ZHANG et al. Protein interacting with C alpha kinase 1 (PICK1) is involved in promoting tumor growth and correlates with poor prognosis of human breast cancer. Cancer Sci. 2010 June; 101(6):1536-42.

1-19. (canceled)

20. A PICK1 inhibitor comprising a peptide portion and a non-peptide portion, wherein the peptide portion consists of:
a) a first peptide comprising an amino acid sequence of the general formula: X₁X₂X₃X₄X₅; and
b) a second peptide comprising an amino acid sequence of the general formula: X₁X₂X₃X₄X₅;
wherein
X₁ is H, N, F, or T, or is absent;
X₂ is W, S, E, or Y; or is absent;
X₃ is L, V, or I;
X₄ is K, I, or R; and
X₅ is V;
and wherein the non-peptide portion comprises:
c) a linker linking the first peptide to the second peptide, and
d) a lipophilic aliphatic group.

21. The PICK1 inhibitor according to claim 20, wherein the first and the second peptide have a length in the range of 3 to 5 amino acid residues.

22. The PICK1 inhibitor according to claim 20, wherein the first and the second peptide are selected from the group consisting of HWLKV (SEQ ID NO: 54), FEIRV (SEQ ID NO: 34), NSIIV (SEQ ID NO: 5), NSVRV (SEQ ID NO: 8), NSLRV (SEQ ID NO: 53), NSIRV (SEQ ID NO: 6), NYIIV (SEQ ID NO: 13), NYIRV (SEQ ID NO: 14), TSIRV (SEQ ID NO: 18), YIIV (SEQ ID NO: 49), SVRV (SEQ ID NO: 44), EIRV (SEQ ID NO: 46), LRV, IIV, VRV, and IRV.

23. The PICK1 inhibitor according to claim 20, wherein the first and the second peptide are selected from the group consisting of HWLKV (SEQ ID NO: 54), NSVRV (SEQ ID NO: 8), SVRV (SEQ ID NO: 44), and LRV.

24. The PICK1 inhibitor according to claim 20, wherein the linker is an NPEG linker.

25. The PICK1 inhibitor according to claim 20, wherein the linker is an NPEG linker which comprises in the range of 1 to 24 ethylene glycol moieties wherein one or more of the backbone oxygen atoms is replaced with a nitrogen atom.

26. The PICK1 inhibitor according to claim 20, wherein the linker is an NPEG-linker which comprises 4 ethylene glycol moieties wherein one or more of the backbone oxygen atoms is replaced with a nitrogen atom.

27. The PICK1 inhibitor according to claim 20, wherein the linker has the structure according to formula (III):

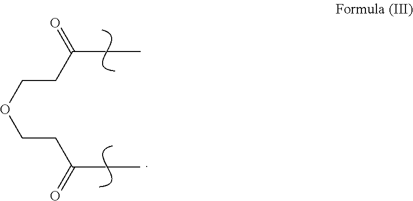

28. The PICK1 inhibitor according to claim 20, wherein the lipophilic aliphatic group is an aliphatic branched chain, an aliphatic unbranched chain, an saturated chain or an unsaturated chain or an aliphatic cycle.

29. The PICK1 inhibitor according to claim 20, wherein the lipophilic aliphatic group is gonane or steroid.

30. The PICK1 inhibitor according to claim 20, wherein the lipophilic aliphatic group is cholesterol.

31. The PICK1 inhibitor according to claim 20, wherein the lipophilic aliphatic group is a C₄-C₂₆ fatty acid or a C₁₄ to C₁₆ fatty acid.

32. The PICK1 inhibitor according to claim 20, wherein the lipophilic aliphatic group is selected from the group consisting of capric acid, lauric acid, myristic acid, palmitic acid, margaric acid, and stearic acid.

33. The PICK1 inhibitor according to claim 20, wherein the lipophilic aliphatic group is myristic acid.

34. The PICK1 inhibitor according to claim 20, wherein the non-peptide portion further comprises one amino acid selected from the group consisting of Asp, β-Asp, β-Ser, β-homo-Ser, and β-Lys.

35. The PICK1 inhibitor according to claim 20, further comprising a detectable moiety.

36. The PICK1 inhibitor according to claim 20, wherein said PICK1 inhibitor has the generic structure of formula (I): 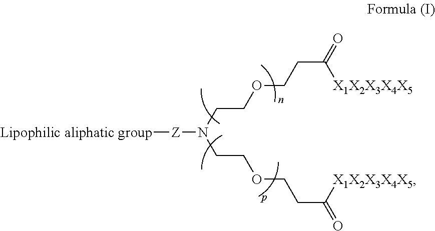
wherein
Z is a bond or a single amino acid;
n is an integer 0 to 12;
p is an integer 0 to 12.

37. A micelle comprising a PICK1 inhibitor comprising
a) a first peptide comprising an amino acid sequence of the general formula: X₁X₂X₃X₄X₅ (SEQ ID NO: 1); and
b) a second peptide comprising an amino acid sequence of the general formula: X₁X₂X₃X₄X₅ (SEQ ID NO: 1);
wherein:
X₁ is H, N, F, or T, or is absent;
X₂ is W, S, E, or Y; or is absent;
X₃ is L, V, or I;
X₄ is K, I, or R; and
X₅ is V;
c) a linker linking the first peptide to the second peptide, and
d) a lipophilic aliphatic group.

38. A method for treatment of a disease or disorder selected from the group consisting of pain, drug addiction, amyotrophic lateral sclerosis, epilepsy, tinnitus, migraine, cancer, ischemia, Alzheimer's disease, or Parkinson's disease, said method comprising administration of a PICK1 inhibitor comprising a peptide portion and a non-peptide portion, wherein the peptide portion consists of
a) a first peptide comprising an amino acid sequence of the general formula: X₁X₂X₃X₄X₅; and
b) a second peptide comprising an amino acid sequence of the general formula: X₁X₂X₃X₄X₅;
wherein
X₁ is H, N, F, or T, or is absent;
X₂ is W, S, E, or Y; or is absent;
X₃ is L, V, or I;
X₄ is K, I, or R; and
X₅ is V;
and wherein the non-peptide portion comprises:
c) a linker linking the first peptide to the second peptide, and
d) a lipophilic aliphatic group,
to an individual in need thereof.

39. The method of claim 38, wherein the pain is mechanical or thermal allodynia or hyperalgesia, or wherein the pain is inflammatory pain.
